SEQUENCE LISTING (5 sequences)

SEQ ID NO: 1 - protein, 328 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 2 - DNA, 2542 bp, Homo sapiens
gaggagggacctacaaagactggaaactattcttagctccgtcactgactccaagttcatcccctctgtctttcagtttggttgagatataggctactcttcccaactcagtcttgaagagtatcaccaactgcctcatgtgtggtgaccttcactgtcgtatgccagtgactcatctggagtaatctcaacaacgagttaccaatacttgctcttgattgataaacagaatggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaagtaaggagtgtttctgagactttgatcacctgaactttctctagcaagtgtaagcagaatggagtgtggttccaagagatccatcaagacaatgggaatggcctgtgccataaaatgtgcttctcttcttcgggatgttgtttgctgtctgatctttgtagactgttcctgtttgctgggagcttctctgctgcttaaattgttcgtcctcccccactccctcctatcgttggtttgtctagaacactcagctgcttctttggtcatccttgttttctaactttatgaactccctctgtgtcactgtatgtgaaaggaaatgcaccaacaaccgtaaactgaacgtgttcttttgtgctcttttataacttgcattacatgttgtaagcatggtccgttctatacctttttctggtcataatgaacactcattttgttagcgagggtggtaaagtgaacaaaaaggggaagtatcaaactactgccatttcagtgagaaaatcctaggtgctactttataataagacatttgttaggccattcttgcattgatataaagaaatacctgagactgggtgatttatatgaaaagaggtttaattggctcacagttctgcaggctgtatgggaagcatggcggcatctgcttctggggacacctcaggagctttactcatggcagaaggcaaagcaaaggcaggcacttcacacagtaaaagcaggagcgagagagaggtgccacactgaaacagccagatctcatgagaagtcactcactattgcaaggacagcatcaaagagatggtgctaaaccattcatgatgaactcacccccatgatccaatcacctcccaccaggctccacctcgaatactggggattaccattcagcatgagatttgggcaggaacacagacccaaaccataccacacacattatcattgttaaactttgtaaagtatttaaggtacatggaacacacgggaagtctggtagctcagcccatttctttattgcatctgttattcaccatgtaattcaggtaccacgtattccagggagcctttcttggccctcagtttgcagtatacacactttccaagtactcttgtagcatcctgtttgtatcatagcactggtcacattgccttacctaaatctgtttgacagtctgctcaacacgactgcaagctccatgagggcagggacatcatctcttccatctttgggtccttagtgcaatacctggcagctagccagtgctcagctaaatatttgttgactgaataaatgaatgcacaaccaaaaaaaaaaaaaaaaaaaaaaaaaaaaaaaaaaaaa

SEQ ID NO: 3 - protein, 556 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 4 - DNA, 2058 bp, Homo sapiens
aaagagaggctggctgttgtatttagtaaagctataaagctgtaagagaaattggctttctgagttgtgaaactgtgggcagaaagttgaggaagaaagaactcaagtacaacccaatgaggttgagatataggctactcttcccaactcagtcttgaagagtatcaccaactgcctcatgtgtggtgaccttcactgtcgtatgccagtgactcatctggagtaatctcaacaacgagttaccaatacttgctcttgattgataaacagaatggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaattgatcatcatagcatctactgcataattgcagtatgtagtgtatttttaatgctaatcaatgtcctggttatcatcctaaaaatgttctggattgaggccactctgctctggagagacatagctaaaccttacaagactaggaatgatggaaagctctatgatgcttatgttgtctacccacggaactacaaatccagtacagatggggccagtcgtgtagagcactttgttcaccagattctgcctgatgttcttgaaaataaatgtggctataccttatgcatttatgggagagatatgctacctggagaagatgtagtcactgcagtggaaaccaacatacgaaagagcaggcggcacattttcatcctgacccctcagatcactcacaataaggagtttgcctacgagcaggaggttgccctgcactgtgccctcatccagaacgacgccaaggtgatacttattgagatggaggctctgagcgagctggacatgctgcaggctgaggcgcttcaggactccctccagcatcttatgaaagtacaggggaccatcaagtggagggaggaccacattgccaataaaaggtccctgaattctaaattctggaagcacgtgaggtaccaaatgcctgtgccaagcaaaattcccagaaaggcctctagtttgactcccttggctgcccagaagcaatagtgcctgctgtgatgtgcaaaggcatctgagtttgaagctttcctgacttctcctagctggcttatgcccctgcactgaagtgtgaggagcaggaatattaaagggattcaggcctc

SEQ ID NO: 5 - protein, 76 aa, Homo sapiens
HPLGSPGSDSDLETSGLQEQRNHLQGKLSELQVEQTSLEPLQESPRPTGVWKSREVATEGIRGHRKMVLYTLRAPR

(19) United States
(12) Patent Application Publication
(10) Pub. No.: US 2015/0199491 A1
(43) Pub. Date: Jul. 16, 2015

(54) METHODS AND SYSTEMS FOR DETERMINING RISK OF HEART FAILURE

(71) Applicant: CRITICAL CARE DIAGNOSTICS, INC., SAN DIEGO, CA (US)

(72) Inventors: James V. Snider, San Diego, CA (US); Robert W. Gerwien, Newington, CT (US)

(21) Appl. No.: 14/592,961

(22) Filed: Jan. 9, 2015

Related U.S. Application Data
(60) Provisional application No. 61/925,877, filed on Jan. 10, 2014.

(51) Int. Cl. G06F 19/00; A61K 33/00

(52) U.S. Cl. CPC G06F 19/3437; A61K 33/00

(57) ABSTRACT

Provided are methods, algorithms, nomograms, and computer/software systems that can be used to accurately determine the risk of developing heart failure within a specific time period in a subject not diagnosed or presenting with heart failure. Also provided are methods, algorithms, nomograms, computer/software systems for selecting a treatment for a subject and determining the efficacy of a treatment for reducing the risk of heart failure in a subject.

### CLAIM OF PRIORITY

This application claims benefit of prior U.S. Provisional Application 61/925,877, filed on Jan. 10, 2014, and is incorporated by reference in its entirety.

### TECHNICAL FIELD

Described herein are methods, systems, and nomograms for determining a subject's risk of developing heart failure, and methods of treating a subject based on their determined risk. The invention relates to the field of cardiovascular medicine and molecular biology.

### BACKGROUND

Heart failure happens when the heart cannot pump enough blood and oxygen to support other organs. Around 5.7 million people in the U.S. have heart failure (Roger et al., Circulation 125:e2-e220, 2013), and heart failure is the primary cause of more than 55,000 deaths each year (Kochanek et al., National Vital Statistics Reports 60(3), 2011). Heart failure is also mentioned as a contributing cause in more than 280,000 deaths (1 in 9 deaths) in 2008 (Roger et al., Circulation 125:e2-e220, 2013). Heart failure costs the U.S. $34.4 billion each year (Heidenriech et al., Circulation 123:933-944, 2011). Early diagnosis and treatment can improve the quality of life and life expectancy for people who have heart failure. Treatment of heart failure usually involves taking medications, reducing salt in the diet, and making other lifestyle adjustments, such as participating in regular physical activity.

Growth stimulation expressed gene 2 (ST2), also known as Interleukin 1 Receptor-Like 1 (IL1RL1) is an interleukin-1 receptor family member with transmembrane (ST2L) and soluble isoforms (sST2 or soluble ST2) (Iwahana et al., Eur. J. Biochem. 264:397-406, 1999). The relationship of ST2 to inflammatory diseases is described in several publications (Arend et al., Immunol. Rev. 223:20-38, 2008; Kakkar et al., Nat. Rev. Drug Discov. 7:827-840, 2008; Hayakawa et al., J. Biol. Chem. 282:26369-26380, 2007; Trajkovic et al., Cytokine Growth Factor Rev. 15:87-95, 2004). Circulating concentrations of human soluble ST2 are elevated in patients suffering from various disorders associated with an abnormal type-2 T helper cell (Th2) response, including systemic lupus erythematosus and asthma, as well as in inflammatory conditions that are mainly independent of a Th2 response, such as septic shock or trauma (Trajkovic et al., Cytokine Growth Factor Rev. 15:87-95, 2004; Brunner et al., Intensive Care Med. 30:1468-1473, 2004). Furthermore, interleukin-33/ST2L signaling represents a crucial cardioprotective mechanism in case of mechanical overload (Seki et al., Circulation Heart Fail. 2:684-691, 2009; Kakkar et al., Nat. Rev. Drug Discov. 7:827-40, 2008; Sanada et al., J. Clin. Invest. 117:1538-1549, 2007). An elevation in human soluble ST2 is also predictive of worse prognosis in patients with heart failure (HF) and those with myocardial infarction (Kakkar et al., Nat. Rev. Drug Discov. 7:827-40, 2008; Weinberg et al., Circulation 107:721-726, 2003; Shimpo et al., Circulation 109:2186-2190, 2004; Januzzi et al., J. Am. Coll. Cardiol. 50:607-613, 2007; Mueller et al., Clin. Chem. 54:752-756, 2008; Rehman et al., J. Am. Coll. Cardiol. 52:1458-65, 2008; Sabatine et al., Circulation 117:1936-1944, 2008).

### SUMMARY

The present invention is based, at least in part, on the development of new methods, algorithms, nomograms, and computer/software systems that can be used to accurately determine the risk of developing heart failure within a specific time period (e.g., within 5 years or within 10 years) in a subject, e.g., a subject not diagnosed or presenting with heart failure. The following describes some specific embodiments of the general invention, but are not intended to be generally limiting.

In some embodiments, the new methods, algorithms, nomograms, and computer/software systems can include one or more, or all of the following: a step of determining a subject's risk of developing heart failure within a specific time period by: providing a set of three or more factors (e.g., four, five, six, seven, or eight) relating to the subject's health selected from the group consisting of: presence or absence of hypertension in the subject, presence or absence of coronary artery disease in the subject, smoking or non-smoking behavior of the subject, body mass index of the subject, serum level of soluble ST2 in the subject, serum level of N-terminal pro-brain natriuretic peptide (NT-proBNP) in the subject, age of the subject, and presence or absence of diabetes in the subject; determining a separate point value for each of the provided factors; adding the separate point values for each of the provided factors together to yield a total points value; and determining the subject's risk of developing heart failure within a specific time period by correlating the total point value with a value on a predictor scale of risk of developing heart failure within the specific time period based on the set of factors obtained from a population of subjects not diagnosed or presenting with heart failure (e.g., a population of subjects not diagnosed, having, or presenting with any other disease as described herein). In any of the methods, algorithms, nomograms, and computer/software systems described herein, the set of factors relating to the subject's health can comprise, consist, or consist essentially of one, two, three, or all four of: (i) presence or absence of hypertension in the subject, smoking or non-smoking behavior of the subject, serum level of soluble ST2 in the subject, age of the subject, body mass index of the subject, and presence or absence of diabetes in the subject; (ii) presence or absence of hypertension in the subject, presence or absence of coronary artery disease in the subject, smoking or non-smoking behavior of the subject, serum level of soluble ST2 in the subject, age of the subject, body mass index of the subject, and presence or absence of diabetes in the subject; (iii) presence or absence of hypertension in the subject, presence or absence of coronary artery disease in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject; and/or (iv) presence or absence of hypertension in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject.

In view of the provided methods, algorithms, nomograms, and computer/software systems, also provided herein are methods of determining the efficacy of a treatment for reducing the risk of developing heart failure in a subject, methods for selecting a treatment for a subject not diagnosed or presenting with heart failure, nomograms for the graphic representation of quantitative probability that a subject not diagnosed or presenting with heart failure will develop heart failure within a specific time period, and computer systems/programs for determining a subject's risk of developing heart failure within a specific period of time, for selecting a treatment for a subject, and for determining the efficacy of treatment for reducing the risk of developing heart failure in a subject.

Provided herein are methods for determining the risk of developing heart failure within a specific time period in a subject not diagnosed or presenting with heart failure that can include one or more of: (a) providing a set of factors relating to the subject's health comprising some or all of: presence or absence of hypertension in the subject, smoking or non-smoking behavior of the subject, serum level of soluble ST2 in the subject, age of the subject, body mass index of the subject, and presence or absence of diabetes in the subject; (b) determining a separate point value for each of the provided factors in (a); (c) adding the separate point values for each of the provided factors in (b) together to yield a total points value; and/or (d) determining the subject's risk of developing heart failure within a specific time period by correlating the total points value in (c) with a value on a predictor scale of risk of developing heart failure within the specific time period based on the set of factors obtained from a population of subjects not diagnosed or presenting with heart failure. Also provided are methods for determining the risk of developing heart failure within a specific time period in a subject not diagnosed or presenting with heart failure that can include one or more of: (a) providing a set of factors relating to the subject's health comprising: presence or absence of hypertension in the subject, presence or absence of coronary artery disease in the subject, smoking or non-smoking behavior of the subject, serum level of soluble ST2 in the subject, age of the subject, body mass index of the subject, and presence or absence of diabetes in the subject; (b) determining a separate point value for each of the provided factors in (a); (c) adding the separate point values for each of the provided factors in (b) together to yield a total points value; and/or (d) determining the subject's risk of developing heart failure within a specific time period by correlating the total points value in (c) with a value on a predictor scale of risk of developing heart failure within the specific time period based on the set of factors obtained from a population of subjects not diagnosed or presenting with heart failure.

Also provided are methods for determining the risk of developing heart failure within a specific time period in a subject not diagnosed or presenting with heart failure that can include one or more of: (a) providing a set of factors relating to the subject's health comprising some or all of: presence or absence of hypertension in the subject, presence or absence of coronary artery disease in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject; (b) determining a separate point value for each of the provided factors in (a); (c) adding the separate point values for each of the provided factors in (b) together to yield a total points value; and/or (d) determining the subject's risk of developing heart failure within a specific time period by correlating the total points value in (c) with a value on a predictor scale of risk of developing heart failure within the specific time period based on the set of factors obtained from a population of subjects not diagnosed or presenting with heart failure.

Also provided are methods for determining the risk of developing heart failure within a specific time period in a subject not diagnosed or presenting with heart failure that can include one or more of: (a) providing a set of factors relating to the subject's health comprising some or all of: presence or absence of hypertension in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject; (b) determining a separate point value for each of the provided factors in (a); (c) adding the separate point values for each of the provided factors in (b) together to yield a total points value; and/or (d) determining the subject's risk of developing heart failure within a specific time period by correlating the total points value in (c) with a value on a predictor scale of risk of developing heart failure within the specific time period based on the set of factors obtained from a population of subjects not diagnosed or presenting with heart failure.

In some embodiments of any of the methods described herein, the providing in (a) includes obtaining the set of factors from the subject's recorded clinical information, e.g., where the obtaining is performed through a computer software program. In some embodiments of any of the methods described herein, the providing in (a) includes the manual entry of the set of factors into a website interface or a software program, e.g., where manual entry is performed by the subject or a health care professional. Some embodiments of any of the methods described herein further include determining one or more of the set of factors in (a) in a subject.

In some embodiments of any of the methods described herein, the presence of hypertension in a subject is characterized as one or both of systolic pressure of ≧140 mm Hg and diastolic pressure of ≧90 mm Hg. Some embodiments of any of the methods described herein include recording the subject's determined risk into the subject's medical file or record, e.g., where the subject's medical file or record is stored in a computer readable medium. In some embodiments of any of the methods described herein, the determining one or both of (b) and (d) is performed using a nomogram. In some embodiments of any of the methods described herein, one or more of the determining in (b), the adding in (c), and the determining in (d) is performed using a software program. In some embodiments of any of the methods described herein, the specific time period is between about 1 year and about 10 years, e.g., 5 years or 10 years.

Some embodiments of any of the methods described herein further include: (e) comparing the determined risk of developing heart failure within the specific time period to a predetermined risk value; (f) identifying a subject whose determined risk of developing heart failure within the specific time period is elevated as compared to the predetermined risk value; and (g) administering a treatment for reducing the risk of developing heart failure to the identified subject, e.g., where one or both of the comparing in (e) and the identifying in (f) are performed using a software program. In some embodiments of any of the methods described herein, the treatment for reducing the risk of developing heart failure is selected from the group of: an anti-inflammatory agent, an anti-thrombotic agent, an anti-platelet agent, a fibrinolytic agent, a lipid-reducing agent, a direct thrombin inhibitor, a glycoprotein IIb/IIIa receptor inhibitor, a calcium channel blocker, a beta-adrenergic receptor blocker, a cyclooxygenase-2 inhibitor, and a renin-angiotensin-aldosterone system (RAAS) inhibitor.

Also provided are methods for determining the efficacy of a treatment for reducing the risk of developing heart failure in a subject that can include one or more of: (a) providing a set of factors relating to the subject's health at a first time point comprising some or all of: presence or absence of hypertension in the subject, smoking or non-smoking behavior of the subject, serum level of soluble ST2 in the subject, age of the subject, body mass index of the subject, and presence or absence of diabetes in the subject; (b) determining a separate point value for each of the provided factors in (a); (c) adding the separate point values for each of the provided factors in (b) together to yield a total points value; (d) determining the subject's risk of developing heart failure within a specific time period at the first time point by correlating the total points value of (c) with a value on a predictor scale of risk of developing heart failure within the specific time period based on the set of factors obtained from a population of subjects not diagnosed or presenting with heart failure; (e) providing a set of factors relating to the subject's health at a second time point comprising: presence or absence of hypertension in the subject, smoking or non-smoking behavior of the subject, serum level of soluble ST2 in the subject, age of the subject, body mass index of the subject, and presence or absence of diabetes in the subject; (f) determining a separate point value for each of the provided factors in (e); (g) adding the separate point values for each of the provided factors in (f) together to yield a total points value; (h) determining the subject's risk of developing heart failure within the specific time period at the second time point by correlating the total points value of (g) with a value on a predictor scale of risk of developing heart failure within the specific time period based on the set of factors obtained from a population of subjects not diagnosed or presenting with heart failure, wherein the second time point is after the first time point, and the subject has received at least two doses of a treatment after the first time point and before the second time point; (i) comparing the subject's risk of developing heart failure within the specific time period determined at the second time point to the subject's risk of developing heart failure within the specific time period determined at the first time point; and/or (j) identifying the treatment administered to a subject having a decreased risk of developing heart failure within the specific time period determined at the second time point as compared the subject's risk of developing heart failure within the specific time period determined at the first time point as being effective for reducing the risk of developing heart failure, or identifying the treatment administered to a subject having an elevated or about the same risk of developing heart failure within the specific time period determined at the second time point as compared to the subject's risk of developing heart failure within the specific time period determined at the first time point as not being effective for reducing the risk of developing heart failure. In some embodiments of any of the methods described herein, one or both of the providing in (a) and the providing in (e) includes obtaining the set of factors from a subject's recorded clinical information, e.g., where the obtaining is performed through a computer software program. In some embodiments of any of the methods described herein, one or both of the providing in (a) and the providing in (e) include the manual entry of the set of factors into a website interface or a software program, e.g., where the manual entry is performed by the subject or by a health care professional. Some embodiments of any of the methods described herein, further include determining one or more of the set of factors in the subject at one or both of the first and second time points. In some embodiments of any of the methods described herein, the presence of hypertension in a subject is characterized as one or both of systolic pressure of ≧140 mm Hg and diastolic pressure of ≧90 mm Hg. Some embodiments of any of the methods described herein further include recording the determined efficacy of the treatment into the subject's medical file or record, e.g., where the subject's medical file or record is stored in a computer readable medium. In some embodiments of any of the methods described herein, the determining in one or both of (b) and (d), and/or the determining in one or both of (f) and (h) is performed using a nomogram. In some embodiments of any of the methods described herein, one or more of the determining in (b), the adding in (c), and the determining in (d) is performed using a software program and/or one or more of the determining in (f), the adding in (g), and the determining in (h) is performed using a software program. In some embodiments of any of the methods described herein, one or both of the comparing in (i) and the identifying in (j) is performed using a software program. In some embodiments of any of the methods described herein, the specific time period is between about 1 year to about 10 years, e.g., 5 years or 10 years. Some embodiments further include administering a treatment for reducing the risk of developing heart failure to the identified subject after the first time point and before the second time point. In some embodiments of any of the methods described herein, the treatment is administration of at least two doses of an agent selected from the group of: an anti-inflammatory agent, an anti-thrombotic agent, an anti-platelet agent, a fibrinolytic agent, a lipid-reducing agent, a direct thrombin inhibitor, a glycoprotein IIb/IIIa receptor inhibitor, a calcium channel blocker, a beta-adrenergic receptor blocker, a cyclooxygenase-2 inhibitor, and a renin-angiotensin-aldosterone system (RAAS) inhibitor.

In some embodiments of any of the methods described herein, the RAAS inhibitor is selected from the group of: an angiotensin-converting enzyme (ACE) inhibitor, an angiotensin II receptor blocker (ARB), aldosterone antagonists, an angiotensin II receptor antagonist, an agent that activates the catabolism of angiotensin II, and an agent that prevents the synthesis of angiotensin I. In some embodiments of any of the methods described herein the lipid-reducing agent is selected from the group of: gemfibrozil, cholestyramine, colestipol, nicotinic acid, probucol, lovastatin, fluvastatin, simvastatin, atorvastatin, pravastatin, and cerivastatin. In some embodiments of any of the methods described herein, the treatment is selected from exercise therapy, smoking cessation therapy, and nutritional consultation.

Also provided are methods for selecting a treatment for a subject not diagnosed or presenting with heart failure that can include one or more of: (a) providing a set of factors relating to the subject's health at a first time point including some or all of: presence or absence of hypertension in the subject, smoking or non-smoking behavior of the subject, serum level of soluble ST2 in the subject, age of the subject, body mass index of the subject, and presence or absence of diabetes in the subject; (b) determining a separate point value for each of the provided factors in (a); (c) adding the separate point values for each of the provided factors in (b) together to yield a total points value; (d) determining the subject's risk of developing heart failure within a specific time period at the first time point by correlating the total points value of (c) with a value on a predictor scale of risk of developing heart failure within the specific time period based on the set of factors obtained from a population of subjects not diagnosed or presenting with heart failure; (e) providing a set of factors relating to the subject's health at a second time point comprising: presence or absence of hypertension in the subject, smoking or non-smoking behavior of the subject, serum level of soluble ST2 in the subject, age of the subject, body mass index of the subject, and presence or absence of diabetes in the subject; (f) determining a separate point value for each of the provided factors in (e); (g) adding the separate point values for each of the provided factors in (f) together to yield a total points value; (h) determining the subject's risk of developing heart failure within the specific time period at the second time point by correlating the total points value of (g) with a value on a predictor scale of risk of developing heart failure within the specific time period based on the set of factors obtained from a population of subjects not diagnosed or presenting with heart failure, wherein the second time point is after the first time point, and the subject has received a treatment after the first time point and before the second time point; (i) comparing the subject's risk of developing heart failure within the specific time period determined at the second time point to the subject's risk of developing heart failure within the specific time period determined at the first time point; and/or (j) identifying a subject having an elevated or about the same risk of developing heart failure within the specific time period determined at the second time point as compared to the subject's risk of developing heart failure within the specific time period determined at the first time point, and selecting an alternate treatment for the subject, or identifying a subject having a reduced risk of developing heart failure within the specific time period determined at the second time point as compared to the subject's risk of developing heart failure within the specific time period determined at the first time point, and selecting the same treatment for the subject. In some embodiments of any of the methods described herein, one or both of the providing in (a) and the providing in (e) includes obtaining the set of factors from a subject's recorded clinical information, e.g., where the obtaining is performed through a computer software program. In some embodiments of any of the methods described herein, one or both of the providing in (a) and the providing in (e) includes the manual entry of the set of factors into a website interface or a software program, e.g., where the manual entry is performed by the subject or by a health care professional. Some embodiments of any of the methods described herein further include determining one or more of the set of factors in a subject at one or both of the first time point and the second time point. In some embodiments of any of the methods described herein, the presence of hypertension in a subject is characterized as one or both of systolic pressure of ≧140 mm Hg and diastolic pressure of ≧90 mm Hg. Some embodiments of any of the methods described herein further include recording the selected treatment into the subject's medical file or record, e.g., where the subject's medical file or record is stored in a computer readable medium. In some embodiments of any of the methods described herein, one or both of the determining in (b) and (d), and/or one or both of the determining in (f) and (h) is performed using a nomogram. In some embodiments of any of the methods described herein, one or more of the determining in (b), the adding in (c), and the determining in (d) is performed using a software program and/or one or more of the determining in (f), the adding in (g), and the determining in (h) is performed using a software program. In some embodiments of any of the methods described herein, one or more of the comparing in (i), the identifying in (j), and the selecting in (j) are performed using a software program. In some embodiments of any of the methods described herein, the specific time period is between about 1 year to 10 years, e.g., 5 years or 10 years. Some embodiments of any of the methods described herein further include administering the selected treatment to the identified subject after the second time point.

Also provided are nomograms for the graphic representation of a quantitative probability that a subject not diagnosed or presenting with heart failure will develop heart failure within a specific time period including the following elements (a), (b), and (c) depicted on a two-dimensional support: (a) a plurality of scales comprising a presence of hypertension scale, a smoking behavior scale, a serum level of soluble ST2 scale, an age of the subject scale, a body mass index scale, and a presence of diabetes scale; (b) a point scale; and (c) a predictor scale, wherein each of the plurality of scales of (a) has values, the plurality of scales of (a) is depicted on the two-dimensional support with respect to the point scale in (b), such that the values on each of the plurality of scales can be correlated with values on the point scale, and the predictor scale contains information correlating a sum of each of correlated values on the point scale to the quantitative probability that a subject not diagnosed or presenting with heart failure will develop heart failure within a specific time period.

Also provided are nomograms for the graphic representation of a quantitative probability that a subject not diagnosed or presenting with heart failure will develop heart failure within a specific time period including the following elements (a), (b), and (c) depicted on a two-dimensional support: (a) a plurality of scales comprising a presence of hypertension scale, a presence of coronary artery disease scale, a smoking behavior scale, a serum level of soluble ST2 scale, an age of the subject scale, a body mass index scale, and a presence of diabetes scale; (b) a point scale; and (c) a predictor scale, where each of the plurality of scales of (a) has values, the plurality of scales of (a) is depicted on the two-dimensional support with respect to the point scale in (b), such that the values on each of the plurality of scales can be correlated with values on the point scale, and the predictor scale contains information correlating a sum of each of correlated values on the point scale to the quantitative probability that a subject not diagnosed or presenting with heart failure will develop heart failure within a specific time period.

Also provided are nomograms for the graphic representation of a quantitative probability that a subject not diagnosed or presenting with heart failure will develop heart failure within a specific time period comprising the following elements (a), (b), and (c) depicted on a two-dimensional support: (a) a plurality of scales including a presence of hypertension scale, a presence of coronary artery disease scale, a smoking behavior scale, a serum level of soluble ST2 scale, a serum level of N-terminal pro-brain natriuretic peptide (NT-proBNP) scale, an age of the subject scale, a body mass index scale, and a presence of diabetes scale; (b) a point scale; and (c) a predictor scale, where each of the plurality of scales of (a) has values, the plurality of scales of (a) is depicted on the two-dimensional support with respect to the point scale in (b), such that the values on each of the plurality of scales can be correlated with values on the point scale, and the risk scale contains information correlating a sum of each of correlated values on the point scale to the quantitative probability that a subject not diagnosed or presenting with heart failure will develop heart failure within a specific time period.

Also provided are nomograms for the graphic representation of a quantitative probability that a subject not diagnosed or presenting with heart failure will develop heart failure within a specific time period that can include some or all of the following elements depicted on a two-dimensional support: (a) a plurality of scales comprising a presence of hypertension scale, a presence of smoking behavior scale, a serum level of soluble ST2 scale, a serum level of N-terminal pro-brain natriuretic peptide (NT-proBNP) scale, an age of the subject scale, a body mass index scale, and a presence of diabetes scale; (b) a point scale; and (c) a predictor scale, where each of the plurality of scales of (a) has values, the plurality of scales of (a) is depicted on the two-dimensional support with respect to the point scale in (b), such that the values on each of the plurality of scales can be correlated with values on the point scale, and the risk scale contains information correlating a sum of each of correlated values on the point scale to the quantitative probability that a subject not diagnosed or presenting with heart failure will develop heart failure within a specific time period.

In any of the nomograms described herein, the two-dimensional support can be a card or piece of paper, or a visual screen or display. In any of the nomograms described herein, the specific time period can be between about 1 year and about 10 years, e.g., 1 months, 2 months, 3 months, 4 months, 5 months, 6 months, 7 months, eight months, 9 months, 10 months, 11 months, 1 year, 2 years, 3 years, 4 years, 5 years, 6 years, 7 years, 8 years, 9 years, or 10 years. Also provided are methods of determining the quantitative probability that a subject not diagnosed or presenting with heart failure will develop heart failure within a specific time period including the use of any of the nomograms described herein.

Also provided are computer-implemented methods that include: accessing a set of factors relating to a subject's health, the set of factors representing one or more of: presence or absence of hypertension in the subject, smoking or non-smoking behavior of the subject, presence or absence of coronary artery disease in the subject, serum level of soluble ST2 in the subject, serum level of N-terminal pro-brain natriuretic peptide (NT-proBNP) in the subject, age of the subject, body mass index of the subject, and presence or absence of diabetes in the subject; determining, using a processor, a separate point value for each factor in the set of factors; determining a total points value as a function of the separate point values; and determining the subject's risk of the subject developing heart failure within a specific time period by correlating the total points value with a value on a predictor scale of risk of developing heart failure within the specific time period, respectively, wherein the predictor scale is based on a set of factors obtained from a population of subjects not diagnosed or presenting with heart failure. Some embodiments of any of the methods described herein include presenting the subject's determined risk of developing heart failure on a user interface. In some embodiments of any of the methods described herein, accessing the set of factors further includes obtaining the set of factors from the subject's recorded clinical information. In some embodiments of any of the methods described herein, accessing the set of factors further includes receiving one or more of the factors through a user interface. Some embodiments of any of the methods described herein further include storing the subject's determined risk on a computer readable storage device. Some embodiments of any of the methods described herein further include comparing the subject's determined risk of developing heart failure within the specific time period to a predetermined risk value; and providing an output indicative of the comparison.

By the term “soluble ST2” is meant a soluble protein containing a sequence at least 90% identical (e.g., at least 95%, 96%, 97%, 98%, 99%, or 100% identical) to NCBI Accession No. NP_{—}003847.2 (SEQ ID NO: 1) or a nucleic acid containing a sequence at least 90% identical (e.g., at least 95%, 96%, 97%, 98%, 99%, or 100% identical) to NCBI Accession No. NM_{—}003856.2 (SEQ ID NO: 2).

By the term “elevated” or “increased” is meant a difference, e.g., a statistically significant difference (e.g., an increase) in a determined or measured level (e.g., risk of developing heart failure) compared to a reference level (e.g., risk of developing heart failure in a population of subjects that do not have cardiovascular disease, do not present with one or more symptoms of cardiovascular disease, are not diagnosed with cardiovascular disease, and do not have one or more factors associated with the development or increased risk of heart failure, e.g., any of the factors described herein).

By the term “health care facility” is meant a location where a subject can receive medical care from a health care professional (e.g., a nurse, a physician, or a physician's assistant). Non-limiting examples of health care facilities include hospitals, clinics, and assisted care facilities (e.g., a nursing home).

By the term “inpatient” is meant a subject that is admitted to a medical care facility (e.g., a hospital or an assisted care facility).

By the term “inpatient treatment” is meant the monitoring and/or medical treatment of a subject that is admitted to a health care facility (e.g., a hospital or assisted care facility). For example, a subject receiving inpatient treatment may be administered one or more therapeutic agents by a health care profession or may undergo a medical procedure (e.g., surgery (e.g., organ transplant, heart bypass surgery), angioplasty, imaging (e.g., magnetic resonance imaging, ultrasound imaging, and computer tomography scanning)). In other examples, one or more marker of a disease or the severity of the condition can be periodically measured by a health care professional to assess the severity or progression of disease or the subject's condition.

By the term “treatment for reducing the risk of developing heart failure” is meant the administration of one or more pharmaceutical agents to a subject or the performance of a medical procedure on the body of a subject (e.g., surgery, such as organ transplant or heart surgery) for the purpose of preventing the development of heart failure in a subject, reducing the frequency, severity, or duration of one or more symptoms of heart failure in a subject, treating heart failure in a subject, or reducing one or more of the factors associated with risk of developing heart failure in a subject (e.g., any of the factors associated with risk of developing heart failure described herein). Non-limiting examples of pharmaceutical agents that can be administered to a subject include nitrates, calcium channel blockers, diuretics, thrombolytic agents, digitalis, renin-angiotensin-aldosterone system (RAAS) modulating agents (e.g., beta-adrenergic blocking agents, angiotensin-converting enzyme inhibitors, aldosterone antagonists, renin inhibitors, and angiotensin II receptor blockers), and cholesterol-lowering agents (e.g., a statin). The term therapeutic treatment also includes an adjustment (e.g., increase or decrease) in the dose or frequency of one or more pharmaceutical agents that a subject can be taking, the administration of one or more new pharmaceutical agents to the subject, or the removal of one or more pharmaceutical agents from the subject's treatment plan. Additional examples of treatment for reducing the risk of developing heart failure include exercise therapy, smoking cessation therapy, and nutritional consultation.

As used herein, a “subject” is a mammal, e.g., a human.

As used herein, a “biological sample” includes one or more of blood, serum, plasma, urine, and body tissue. Generally, a biological sample is a sample containing serum, blood, or plasma.

As used herein, the term “antibody” refers to a protein that binds to an antigen and generally contains heavy chain polypeptides and light chain polypeptides. Antigen recognition and binding occurs within the variable regions of the heavy and light chains. A given antibody comprises one of five different types of heavy chains, called alpha, delta, epsilon, gamma, and mu, the categorization of which is based on the amino acid sequence of the heavy chain constant region. These different types of heavy chains give rise to five classes of antibodies, IgA (including IgA1 and IgA2), IgD, IgE, IgG (IgG1, IgG2, IgG3, and IgG4) and IgM, respectively. The term antibody, as used herein, encompasses single domain antibodies, conjugated antibodies (e.g., antibodies conjugated to detectable label, e.g., a particle (such as a metal nanoparticle, e.g., a gold nanoparticle), an enzyme, a fluorophore, a dye, or a radioisotope), and antigen-binding antibody fragments.

As used herein, the term “Th2-associated disease” refers to a disease associated with an abnormal type-2 T helper cell (Th2) response.

As used herein, the term “cardiovascular disease” refers to a disorder of the heart and blood vessels, and includes disorders of the arteries, veins, arterioles, venules, and capillaries.

The term “coronary artery disease” is an art-known term and refers to a cardiovascular condition characterized by plaque build-up along the inner walls of the arteries (e.g., arteries of the heart), which narrow and restricts blood flow of the arteries. Coronary artery disease is also called “atherosclerotic heart disease” in the art. Exemplary methods for determining the presence of coronary artery disease are described herein. Additional methods for determining the presence of coronary artery disease are known in the art.

The term “diabetes” is an art-known term and refers to a group of metabolic diseases in which a subject has elevated blood glucose levels, either because the pancreas does not produce enough insulin or because cells in the body do not respond to the insulin that is produced by the pancreas (a phenomenon described as insulin resistance in the art). Diabetes as used herein refers to both type I diabetes (also called diabetes mellitus, insulin-dependent diabetes mellitus (IDD), and juvenile diabetes in the art) and type II diabetes (also called non-insulin-dependent diabetes mellitus (IDDM) or adult-onset diabetes in the art). Non-limiting methods of diagnosing a subject as having diabetes are described herein. Additional methods of diagnosing a subject as having diabetes are known in the art.

By the term “additional marker” is meant a protein, nucleic acid, lipid, or carbohydrate, or a combination (e.g., two or more) thereof, that is diagnostic or prognostic of the presence of a particular disease (e.g., heart failure). The methods described herein can further include detecting a level of at least one additional marker in a sample from the subject. Several additional markers useful for the diagnosis or prognosis of heart failure are known in the art (e.g., proANP, NT-proANP, ANP, proBNP, NT-proBNP, BNP, troponin, CRP, creatinine, Blood Urea Nitrogen (BUN), liver function enzymes, albumin, and bacterial endotoxin; and those markers described in U.S. Patent Application Nos.: 2007/0248981; 2011/0053170; 2010/0009356; 2010/0055683; 2009/0264779; each of which is hereby incorporated by reference).

By the term “hypertriglyceridemia” is meant a triglyceride level that is greater than or equal to 180 ng/mL (e.g., greater than or equal to 200 ng/mL).

By the term “hypercholesterolemia” is meant an increased level of at least one form of cholesterol or total cholesterol in a subject. For example, a subject with hypercholesterolemia can have a high density lipoprotein (HDL) level of ≧40 mg/dL (e.g., ≧50 mg/dL or ≧60 mg/mL), a low density lipoprotein (LDL) level of ≧130 mg/dL (e.g., ≧160 mg/dL or ≧200 mg/dL), and/or a total cholesterol level of ≧200 mg/dL (e.g., 240 mg/dL).

By the term “hypertension” is meant an increased level of systolic and/or diastolic blood pressure. For example, a subject with hypertension can have a systolic blood pressure that is ≧120 mmHg (e.g., ≧140 mmHg or ≧160 mmHg) and/or a diastolic blood pressure that is ≧80 mmHg (e.g., ≧90 mmHg or ≧100 mmHg).

By the term “healthy subject” is meant a subject that does not have a disease (e.g., cardiovascular disease or pulmonary disease). For example, a healthy subject has not been diagnosed as having a disease and is not presenting with one or more (e.g., two, three, four, or five) symptoms of a disease state.

The term “predictor scale” is an art-known term and means a two-dimensional (e.g., represented on a piece of paper, a screen (e.g., a screen of a computer or personal hand-held electronic device)), or a three-dimensional graphical calculating device (e.g., a projected hologram) that provides a correlation between any specific total point score (e.g., a total point score that is the sum of the individual point scores determined for three or more factors (e.g., four, five, six, or seven) relating to the subject's health (e.g., three or more factors selected from the group of: presence or absence of hypertension in the subject, presence or absence of coronary artery disease in the subject, smoking or non-smoking behavior of the subject, body mass index of the subject, serum level of soluble ST2 in the subject, serum level of N-terminal pro-brain natriuretic peptide (NT-proBNP) in the subject, age of the subject, and presence or absence of diabetes in the subject) and a subject's risk of developing heart failure within a specific time period. A predictor scale can be part of a nomogram (e.g., any of the exemplary nomograms described herein). Exemplary types of predictor scales are described herein.

By the term “nomogram” is meant a graphical calculating device that is a two-dimensional (e.g., a piece of paper, a screen of a computer or personal hand-held electronic device) or three-dimensional (e.g., a projected hologram) graphical calculating device that provides scales for determining a point score for each of three or more (e.g., four, five, six, or seven) factors relating to the subject's health (e.g., three or more factors selected from the group of: presence or absence of hypertension in the subject, presence or absence of coronary artery disease in the subject, smoking or non-smoking behavior of the subject, body mass index of the subject, serum level of soluble ST2 in the subject, serum level of N-terminal pro-brain natriuretic peptide (NT-proBNP) in the subject, age of the subject, and presence or absence of diabetes in the subject), and a predictor scale that provides a correlation between a total point score (e.g., a total point score that is the sum of the individual point scores determined for the three or more factors relating to the subject's health) and a subject's risk of developing heart failure within a specific time period.

Unless otherwise defined, all technical and scientific terms used herein have the same meaning as commonly understood by one of ordinary skill in the art to which this invention belongs. Methods and materials are described herein for use in the present invention. Other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a summary of the analysis of an exemplary seven parameter model, Model 1.

FIG. 2 is a set of graphs showing the effect each of soluble ST2, presence or absence of diabetes, presence or absence of hypertension, presence or absence of smoking, age, BMI, and presence or absence of coronary artery disease on heart failure-free survival.

FIG. 3 is a graph showing the partial χ² statistics of the association of soluble ST2, presence or absence of diabetes, presence or absence of hypertension, presence or absence of smoking, age, BMI, and presence or absence of coronary artery disease, with response.

FIG. 4 is a graph showing the bootstrap validation of the calibration curve of an exemplary seven parameter model (Model 1).

FIG. 5 is an exemplary nomogram for determining a subject's probability of heart failure-free survival within a period of 5 years or 10 years, based on an exemplary seven parameter model (Model 1).

FIG. 6 is a summary of the exemplary nomogram based on an exemplary seven parameter model (Model 1).

FIG. 7 is a summary of the analysis of an exemplary six parameter model, Model 2.

FIG. 8 is a set of graphs showing the effect each of presence or absence of hypertension, presence or absence of smoking behavior, serum soluble ST2 levels, age, body mass index, and presence or absence of diabetes on heart failure-free survival.

FIG. 9 is a graph showing the partial χ² statistics of the association of presence or absence of hypertension, presence or absence of smoking behavior, serum soluble ST2 levels, age, body mass index, and presence or absence of diabetes, with response.

FIG. 10 is a graph showing the bootstrap validation of the calibration curve of an exemplary six parameter model, Model 2.

FIG. 11 is an exemplary nomogram for determining a subject's probability of heart failure-free survival within a period of 5 years or 10 years, based on an exemplary six parameter model (Model 2).

FIG. 12 is a summary of an exemplary nomogram based on an exemplary six parameter model (Model 2).

FIG. 13 is a summary of the analysis of an exemplary eight parameter model, Model 3.

FIG. 14 is a set of exemplary graphs showing the effect each of presence or absence of smoking behavior, serum soluble ST2 levels, presence or absence of diabetes, presence or absence of hypertension, serum NT-proBNP levels, age, BMI, and presence or absence of coronary artery disease on heart failure-free survival.

FIG. 15 is an exemplary graph showing the partial χ² statistics of the association of presence or absence of smoking behavior, serum soluble ST2 levels, presence or absence of diabetes, presence or absence of hypertension, serum NT-proBNP levels, age, BMI, and presence or absence of coronary artery disease, with response.

FIG. 16 is a graph showing the bootstrap validation of the calibration curve of an exemplary eight parameter model (Model 3).

FIG. 17 is an exemplary nomogram for determining a subject's probability of heart failure-free survival within a period of 5 years or 10 years, based on an exemplary eight parameter model (Model 3).

FIG. 18 is a summary of the exemplary nomogram based on an exemplary eight parameter model (Model 3).

FIG. 19 is a summary of the analysis of an exemplary seven parameter model (Model 4).

FIG. 20 is a set of exemplary graphs showing the effect each of presence or absence of serum soluble ST2 levels, presence or absence of hypertension, serum NT-proBNP levels, presence or absence of smoking behavior, age, BMI, and presence or absence of diabetes on heart failure-free survival.

FIG. 21 is a graph showing the partial χ² statistics of the association of presence or absence of serum soluble ST2 levels, presence or absence of hypertension, serum NT-proBNP levels, presence or absence of smoking behavior, age, BMI, and presence or absence of diabetes, with response.

FIG. 22 is a graph showing the bootstrap validation of the calibration curve of an exemplary seven parameter model (Model 4).

FIG. 23 is an exemplary nomogram for determining a subject's probability of heart failure-free survival within a period of 5 years or 10 years, based on an exemplary seven parameter model (Model 4).

FIG. 24 is a summary of the exemplary nomogram based on an exemplary seven parameter model (Model 4).

FIG. 25 is a chart providing a comparison of the accuracy of each of exemplary Models 1-4.

FIG. 26A is a block diagram of an exemplary system that can be used for implementing any of the methods described herein.

FIGS. 26B and 26C represent exemplary user interfaces.

FIG. 27 is a schematic diagram of an exemplary environment used for implementing any of the methods described herein.

FIG. 28 is a flowchart that illustrates an exemplary sequence of operations for determining a risk of developing heart failure using any of the methods described herein.

FIG. 29 is a block diagram of an exemplary computer system.

### DETAILED DESCRIPTION

Described herein are methods for determining a subject's risk of developing heart failure within a specific time period, methods of selecting a treatment for a subject, methods for treating a subject, and methods of determining the efficacy of a treatment for reducing the risk of heart failure in a subject. Also provided are nomograms, algorithms, and systems, e.g., computer systems/software, for performing any of the methods described herein. The methods, nomograms, algorithms, and systems, e.g., computer systems/software, described herein are useful in a wide variety of clinical contexts. For example, such methods nomograms, algorithms, and systems can be used for general population screening, including screening by doctors, e.g., in hospitals and outpatient clinics, as well as the emergency room.

Generally, the methods provided herein include a step of determining a subject's risk of developing heart failure within a specific time period by: providing a set of three or more (e.g., six, seven, or eight) factors relating to the subject's health, selected from the group of: presence or absence of hypertension in the subject, presence or absence of coronary artery disease in the subject, smoking or non-smoking behavior of the subject, body mass index of the subject, serum level of soluble ST2 in the subject, serum level of N-terminal pro-brain natriuretic peptide (NT-proBNP) in the subject, age of the subject, and presence or absence of diabetes in the subject; determining a separate point value for each of the provided factors; adding the separate point values for each of the provided factors together to yield a total points value; and determining the subject's risk of developing heart failure within a specific time period by correlating the total point value with a value on a predictor scale of risk of developing heart failure within the specific time period based on the set of factors obtained from a population of subjects not diagnosed or presenting with heart failure.

In any of the methods, algorithms, nomograms, and computer/software systems described herein, the set of factors relating to the subject's health comprises, consists, or consists essentially of one, two, three, or all four of: (i) presence or absence of hypertension in the subject, smoking or non-smoking behavior of the subject, serum level of soluble ST2 in the subject, age of the subject, body mass index of the subject, and presence or absence of diabetes in the subject; (ii) presence or absence of hypertension in the subject, presence or absence of coronary artery disease in the subject, smoking or non-smoking behavior of the subject, serum level of soluble ST2 in the subject, age of the subject, body mass index of the subject, and presence or absence of diabetes in the subject; (iii) presence or absence of hypertension in the subject, presence or absence of coronary artery disease in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject; and/or (iv) presence or absence of hypertension in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject. In some embodiments, the set of factors comprises, consists, or consists essentially of the presence or absence of hypertension in the subject, smoking or non-smoking behavior of the subject, serum level of soluble ST2 in the subject, age of the subject, body mass index of the subject, and presence or absence of diabetes in the subject, with the optional inclusion of the factor(s) of presence or absence of coronary artery disease in the subject and/or serum level of N-terminal pro-brain natriuretic peptide (NT-proBNP).

Various non-limiting aspects of these methods, algorithms, nomograms, and systems are described below.

### ST2

The ST2 gene is a member of the interleukin-1 receptor family whose protein product exists both as a trans-membrane form as well as a soluble receptor that is detectable in serum (Kieser et al., FEBS Lett. 372(2-3):189-193, 1995; Kumar et al., J. Biol. Chem. 270(46):27905-27913, 1995; Yanagisawa et al., FEBS Lett. 302(1):51-53, 1992; Kuroiwa et al., Hybridoma 19(2):151-159, 2000). Soluble ST2 was described to be markedly up-regulated in an experimental model of heart failure (Weinberg et al., Circulation 106(23):2961-2966, 2002), and data suggest that human soluble ST2 concentrations are also elevated in those with chronic severe heart failure (Weinberg et al., Circulation 107(5):721-726, 2003), as well as in those with acute myocardial infarction (Shimpo et al., Circulation 109(18):2186-2190, 2004).

Without wishing to be bound by theory, the transmembrane form of ST2 is thought to play a role in modulating responses of T helper type 2 cells (Lohning et al., Proc. Natl. Acad. Sci. U.S.A. 95(12):6930-6935, 1998; Schmitz et al., Immunity 23(5):479-490, 2005), and may play a role in development of tolerance in states of severe or chronic inflammation (Brint et al., Nat. Immunol. 5(4):373-379, 2004), while the soluble form of ST2 is up-regulated in growth stimulated fibroblasts (Yanagisawa et al., 1992, supra). Experimental data suggest that the ST2 gene is markedly up-regulated in states of cardiomyocyte stretch (Weinberg et al., 2002, supra) in a manner analogous to the induction of the BNP gene (Bruneau et al., Cardiovasc. Res. 28(10):1519-1525, 1994).

Tominaga et al. (FEBS Lett. 258:301-304, 1989) isolated murine genes that were specifically expressed by growth stimulation in BALB/c-3T3 cells. Haga et al. (Eur. J. Biochem. 270:163-170, 2003) describes that the ST2 gene was named on the basis of its induction by growth stimulation. The ST2 gene encodes two protein products: ST2 or sST2 which is a soluble secreted form, and ST2L, a transmembrane receptor form that is very similar to the interleukin-1 receptors. The HUGO Nomenclature Committee designated the human homolog of ST2, the cloning of which was described in Tominaga et al., Biochim. Biophys. Acta. 1171:215-218, 1992, as Interleukin 1 Receptor-Like 1 (IL1RL1). The two terms are used interchangeably herein.

The mRNA sequence of the shorter, soluble isoform of human ST2 can be found at GenBank Acc. No. NM_{—}003856.2 (SEQ ID NO: 2), and the polypeptide sequence is at GenBank Acc. No. NP_{—}003847.2 (SEQ ID NO: 1). The mRNA sequence for the longer form of human ST2 is at GenBank Acc. No. NM_{—}016232.4 (SEQ ID NO: 4), and the polypeptide sequence is at GenBank Acc. No. NP_{—}057316.3 (SEQ ID NO: 3). Additional information is available in the public databases at GeneID: 9173, MIM ID #601203, and UniGene No. Hs. 66. In general, in the methods described herein, the human soluble form of ST2 polypeptide is measured.

Levels of soluble ST2 in a sample of a subject (e.g., any of the samples described herein) can be determined using methods known in the art, e.g., using the anti-soluble human ST2 antibodies described in U.S. Pat. No. 8,420,785, U.S. Patent Application Publication No. 2013/0177931, and WO 2011/127412. Additional antibodies that specifically bind to soluble ST2 are known in the art. The level of soluble ST2 for a subject can be provided by determining the serum level of soluble ST2 (e.g., by performing an assay on a sample containing serum from the subject to determine the level of soluble ST2, e.g., any of the assays described herein) or obtaining the serum level of soluble ST2 from the subject's medical file (e.g., a computer readable medium). In some examples where the serum level of soluble ST2 is determined in a sample containing serum from the subject, the method further includes a step of obtaining or providing a sample containing serum from the subject.

For example, the levels of soluble ST2 in a control healthy subject can be about 18.8 ng/mL or below. In some embodiments, a level of soluble ST2 in a healthy control subject is a range of about 14.5 to about 25.3 ng/mL or a range of about 18.1 to about 19.9 ng/mL. The level of soluble ST2 level in a healthy control female subject can be, e.g., about 16.2 ng/mL or within any of the ranges listed in Table 1. The level of soluble ST2 for a healthy control male subject can be, e.g., about 23.6 ng/mL or within any of the ranges listed in Table 1. A level of soluble ST2 in a healthy control subject (e.g., male or female subject) can be up to about 25.3 ng/mL, or 19.9 ng/mL (for females) or 30.6 ng/mL (for males). As can be appreciated by those skilled in the art, the serum level of soluble ST2 will vary depending on how the serum level of soluble ST2 is determined (e.g., depending on which antibody or pairs of antibodies is/are used for detection in the assay).

TABLE 1
Soluble ST2 Concentrations in U.S. Self-Reported Healthy Cohort

| | Entire Cohort | | Male | | Female | |
|---|---|---|---|---|---|---|
| | ST2 | | ST2 | | ST2 | |
| Percentiles | (ng/mL) | 95% CI | (ng/mL) | 95% CI | (ng/mL) | 95% CI |
| 2.5 | 8.0 | 7.1 to 8.6 | 8.6 | 7.7 to 11.8 | 7.3 | 5.5 to 8.4 |
| 5 | 9.3 | 8.4 to 10.2 | 11.8 | 8.6 to 12.7 | 8.5 | 7.3 to 9.4 |
| 10 | 11.5 | 10.3 to 11.9 | 13.7 | 12.2 to 14.8 | 10.2 | 9.0 to 11.2 |
| 25 | 14.5 | 13.7 to 15.2 | 17.6 | 16.8 to 18.7 | 12.4 | 11.9 to 13.5 |
| median | 18.8 | 18.2 to 19.9 | 23.6 | 21.3 to 25.1 | 16.2 | 15.4 to 17.4 |
| 75 | 25.3 | 23.8 to 26.9 | 30.6 | 28.7 to 33.3 | 19.9 | 18.8 to 20.8 |
| 90 | 34.3 | 32.4 to 35.6 | 37.2 | 35.5 to 40.9 | 23.7 | 22.2 to 25.8 |
| 95 | 37.9 | 35.9 to 41.3 | 45.4 | 39.4 to 48.6 | 29.0 | 24.6 to 33.2 |
| 97.5 | 45.6 | 40.1 to 48.7 | 48.5 | 45.8 to 58.5 | 33.1 | 29.6 to 39.9 |

### NT-proBNP

N-terminal pro-brain natriuretic peptide (NT-proBNP) is a 76 amino-acid N-terminal fragment of brain natriuretic peptide. BNP is synthesized as a 134-amino acid preprohormone (pre-pro-BNP). Removal of the 26-residue N-terminal signal peptide generates the prohormone, proBNP. ProBNP is subsequently cleaved between arginine 102 and serine 103 by a specific convertase into NT-proBNP. The sequence of human NT-proBNP is provided below.

NT-ProBNP
(SEQ ID NO: 5)
hplgspgsas dletsglqeq rnhlqgklse lqveqtslep
lqesprptgv wksrevateg irghrkmvly tlrapr

Levels of NT-proBNP can be determined using assays known in the art, e.g., Stratus® CS Acute Care™ NT-proBNP assay, and Immulite® 2500 NT-proBNP assay. Additional examples of commercially available assays for determining a level of NT-proBNP are known in the art.

The serum level of NT-proBNP in a subject can be provided by determining the level of NT-proBNP in a subject (e.g., performing an assay on a sample containing serum from the subject to determine the level of NT-proBNP). In some examples where an assay is performed to determine the serum level of NT-proBNP, the method further includes a step of obtaining or providing a biological sample containing serum from the subject. In other examples, the serum level of NT-proBNP in a subject can be provided by obtaining the serum level of NT-proBNP from the subject's medical file (e.g., a computer readable medium). As can be appreciated by those skilled in the art, the serum level of soluble NT-proBNP will vary depending on how the serum level of NT-proBNP is determined (e.g., depending on which antibody or pairs of antibodies is/are used for detection in the assay).

### Diabetes

The presence of diabetes in a subject can be determined by, e.g., evaluating a subject's clinical file and/or detecting one or more symptoms of diabetes in a subject. Non-limiting examples of symptoms of diabetes include, e.g., excessive thirst and appetite, increased urination, unusual weight loss or gain, fatigue, nausea, vomiting, blurred vision, vaginal infections, yeast infections, dry mouth, flow-healing of sores or cuts, itching skin (e.g., in groin or vaginal area), ketoacidosis, elevated fasting blood glucose levels, elevated random blood sugar level, decreased oral glucose tolerance, and elevated glycohemoglobin A1c (e.g., elevated glycated hemoglobin levels (HbA1C)). Additional methods of determining the presence of diabetes in a subject or diagnosing a subject as having diabetes are known in the art.

In some embodiments, the providing of the factor regarding the presence or absence of diabetes in a subject includes identifying, determining, or diagnosing a subject as having diabetes, obtaining information regarding the presence or absence of diabetes in a subject from the subject's medical file (e.g., a computer readable medium), or interviewing the subject to request the subject to provide information regarding whether he or she has diabetes.

### Hypertension

Hypertension is meant as an elevated level of systolic and/or diastolic blood pressure. For example, a subject with hypertension can have a systolic blood pressure that is ≧120 mmHg (e.g., ≧140 mmHg or ≧160 mmHg) and/or a diastolic blood pressure that is ≧80 mmHg (e.g., ≧90 mmHg or ≧100 mmHg). Methods for determining systolic and/or diastolic blood pressure are well-known by those skilled in the art.

In some embodiments, the providing of the factor regarding the presence or absence of hypertension in a subject includes identifying or determining that a subject has hypertension, obtaining information regarding the presence or absence of hypertension in a subject from the subject's medical file (e.g., a computer readable medium), or interviewing the subject to request the subject to provide information regarding whether he or she has hypertension or is taking an anti-hypertensive medication.

### Coronary Artery Disease

Coronary artery disease is an art-known term and refers to a type of cardiovascular disease characterized by plaque build-up along the inner walls of the arteries (e.g., arteries of the heart), which narrows and restricts blood flow of the arteries. Coronary artery disease can be determined in a subject, e.g., by the observation of one of more symptoms of coronary artery disease in the subject. Non-limiting symptoms of coronary artery disease include: chest pain, shortness of breath when exercising or during other vigorous activity, fast heartbeat, weakness, dizziness, nausea, and increased sweating. As is well known in the art, coronary artery disease can also be determined in a subject by physical examination (e.g., detection of a bruit using a stethoscope), blood tests (e.g., blood tests to determine the levels of one or more of cholesterol, triglycerides, and glucose in the subject), determining the ankle/brachial index of the subject, and performing electrocardiogram, echocardiography, computed tomography scanning, stress testing, and/or angiography on the subject. Additional exemplary methods for determining the presence of coronary artery disease in a subject are well-known in the art.

In some embodiments, the providing of the factor regarding the presence or absence of coronary artery disease in a subject includes identifying, diagnosing, or determining that a subject has coronary artery disease, obtaining information regarding the presence or absence of coronary artery disease in a subject from the subject's medical file (e.g., a computer readable medium), or interviewing the subject to request the subject to provide information regarding whether he or she has coronary artery disease.

### Body Mass Index

As is well-known in the art, body mass index for a subject is determined using the formula, BMI=mass (kg)/(height (m))². A BMI can be determined for a subject by determining the subject's mass (also sometimes referred to as weight) and height, and calculating the subject's BMI. A BMI can also be determined for a subject by obtaining the subject's mass and height from the subject's clinical file, and calculating the subject's BMI. A subject can also determine his or her own BMI by assessing his or her own mass and height, and calculating his or her own BMI. The subject can also provide (e.g., verbally) a medical professional information regarding his or her mass and height, and the physician can determine the subject's BMI. Additional methods for determining a subject's BMI are known in the art.

In some embodiments, providing the BMI of a subject includes determining the subject's BMI, obtaining information regarding the subject's BMI from the subject's medical file (e.g., a computer readable medium), or interviewing the subject to request the subject to provide information relating to the determination of BMI (e.g., the subject's weight and height). As used herein, “interviewing a subject” can include presenting the subject with questions orally or in writing (e.g., via a paper or digital questionnaire).

### Age

A subject's age can be determined, e.g., by reviewing information in a subject's clinical file and/or interviewing the subject. A subject can also provide information about his or her age to a medical professional orally. A subject's age can also be determined by interviewing family members or checking government records.

In some embodiments, the providing of the factor regarding the age of a subject includes obtaining information regarding the age of the subject from the subject's medical file (e.g., a computer readable medium), or interviewing the subject or the subject's family members to provide information regarding the subject's age.

### Smoking

A subject's smoking behavior can be determined by interviewing (e.g., asking orally or by a questionnaire or computer) the subject or by reviewing the subject's clinical file. A subject who has smoked for a period of greater than 1 month (e.g., greater than two months, three months, four months, five months, six months, seven months, eight months, 9 months, 10 months, 11 months, 12 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, or 60 years) is identified as having smoking behavior (e.g., even if the subject has ceased smoking at the time of the interview). For example, a subject having smoking behavior can have smoked the equivalent of at least 0.1 pack-year, 0.5 pack-year, 0.75 pack-year, 1.0 pack-year, 1.5 pack-years, 2.0 pack-years, 2.5 pack-years, 3.0 pack-years, 3.5 pack-years, 4.0 pack-years, 4.5 pack-years, 5.0 pack-years, 5.5 pack-years, 6.0 pack-years, 7.0 pack-years, 7.5 pack-years, 8.0 pack-years, 8.5 pack-years, 9.0 pack-years, 9.5 pack-years, 10 pack-years, 11 pack-years, 12 pack-years, 13 pack-years, 14 pack-years, 15 pack-years, 16 pack-years, 17 pack-years, 18 pack-years, 19 pack-years, 20 pack-years, 21 pack-years, 22 pack-years, 23 pack-years, 24 pack-years, 25 pack-years, 30 pack-years, 35 pack-years, 40 pack-years, 45 pack-years, 50 pack-years, 55 pack-years, 60 pack-years, 65 pack-years, 70 pack-years, 75 pack-years, or 80 pack-years. A subject can be determined to have present smoking behavior based on the subject's self-identification as a smoker.

In some embodiments, the providing of the factor regarding the presence or absence of smoking behavior in a subject includes determining the presence or absence of smoking behavior in the subject, obtaining information regarding the presence or absence or extent of smoking behavior in a subject from the subject's medical file (e.g., a computer readable medium), or interviewing the subject or the subject's family members regarding the presence or absence or extent of smoking behavior in the subject.

### Nomograms

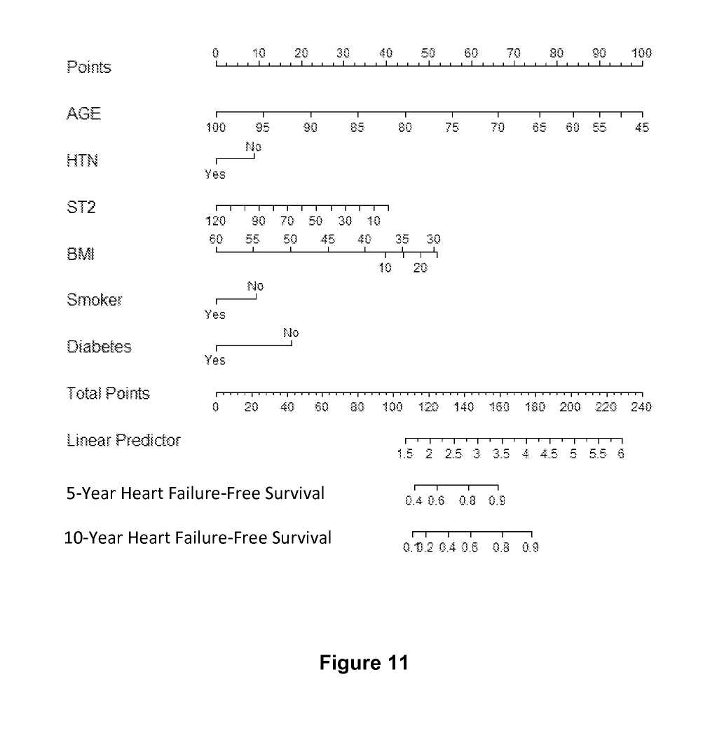

Provided herein are nomograms for the graphic representation of a quantitative probability that a subject not diagnosed or presenting with heart failure will develop heart failure within a specific time period (e.g., within 1 months, 2 months, 3 months, 4 months, 5 months, 6 months, 7 months, eight months, 9 months, 10 months, 11 months, 1 year, 2 years, 3 years, 4 years, 5 years, 6 years, 7 years, 8 years, 9 years, or 10 years). In a first example, such a nomogram can include the following elements depicted on a two-dimensional or three-dimensional support: (a) a plurality of scales including or consisting of a presence of hypertension scale, a smoking behavior scale, a serum level of soluble ST2 scale, an age of the subject scale, a body mass index scale, and a presence of diabetes scale; (b) a point scale; and (c) a predictor scale. An example of one such nomogram is shown in FIG. 11.

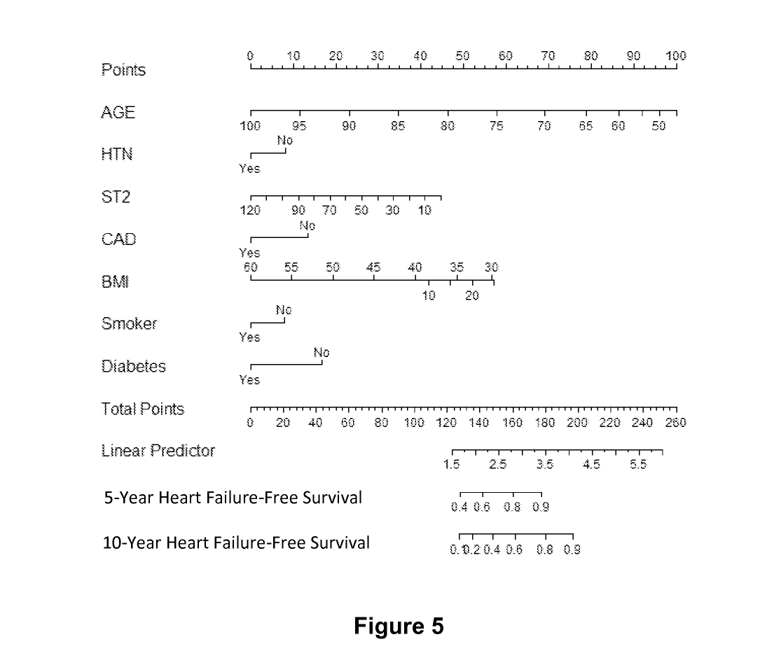

Another example of a nomogram for the graphic representation of a quantitative probability that a subject not diagnosed or presenting with heart failure will develop heart failure within a specific time period (e.g., within 1 months, 2 months, 3 months, 4 months, 5 months, 6 months, 7 months, eight months, 9 months, 10 months, 11 months, 1 year, 2 years, 3 years, 4 years, 5 years, 6 years, 7 years, 8 years, 9 years, or 10 years) includes some or all of the following elements (a), (b), and (c) depicted on a two-dimensional or three-dimensional support: (a) a plurality of scales including or consisting of a presence of hypertension scale, a presence of coronary artery disease scale, a smoking behavior scale, a serum level of soluble ST2 scale, an age of the subject scale, a body mass index scale, and a presence of diabetes scale; (b) a point scale; and (c) a predictor scale. An example of one such nomogram is shown in FIG. 5.

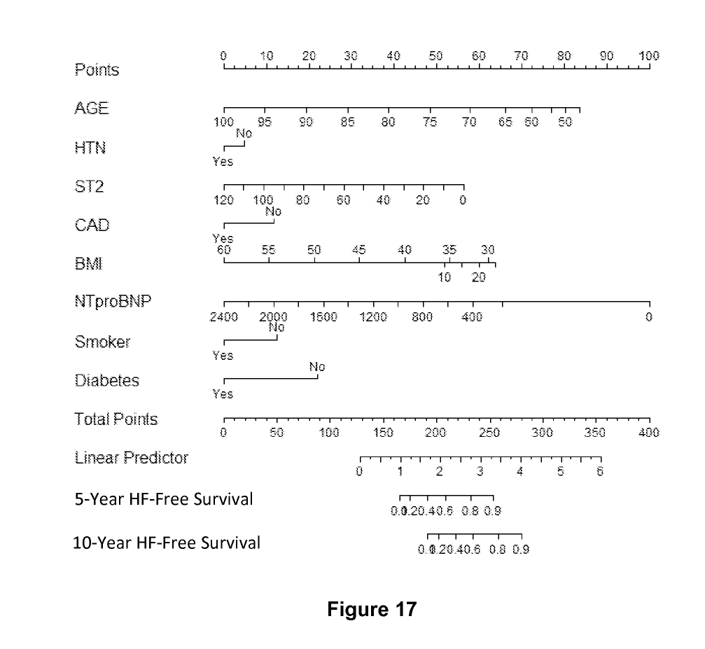

An additional example of a nomogram for the graphic representation of a quantitative probability that a subject not diagnosed or presenting with heart failure will develop heart failure within a specific time period e.g., within 1 months, 2 months, 3 months, 4 months, 5 months, 6 months, 7 months, eight months, 9 months, 10 months, 11 months, 1 year, 2 years, 3 years, 4 years, 5 years, 6 years, 7 years, 8 years, 9 years, or 10 years) includes some or all of the following elements (a), (b), and (c) depicted on a two-dimensional or three-dimensional support: (a) a plurality of scales including or consisting of a presence of hypertension scale, a presence of coronary artery disease scale, a smoking behavior scale, a serum level of soluble ST2 scale, a serum level of N-terminal pro-brain natriuretic peptide (NT-proBNP) scale, an age of the subject scale, a body mass index scale, and a presence of diabetes scale; (b) a point scale; and (c) a predictor scale. An example of one such nomogram is shown in FIG. 17.

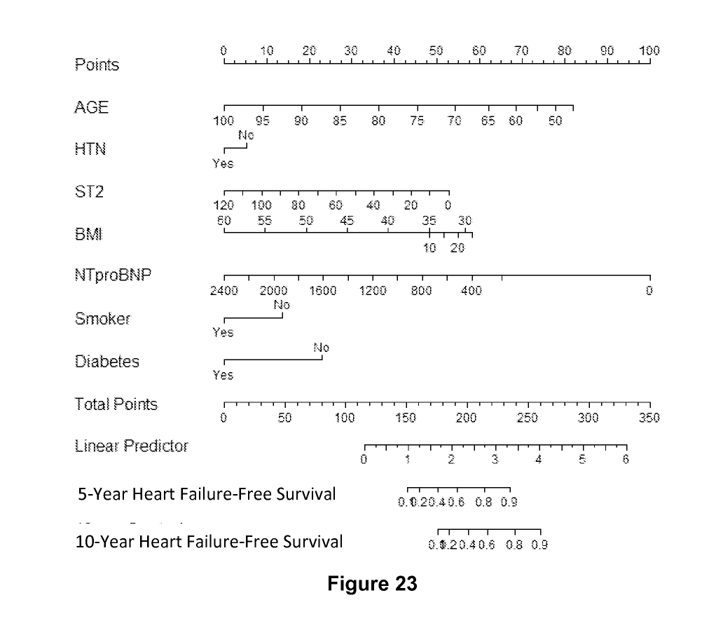

Another example of a nomogram for the graphic representation of a quantitative probability that a subject not diagnosed or presenting with heart failure will develop heart failure within a specific time period includes some or all of the following elements depicted on a two-dimensional or three-dimensional support: (a) a plurality of scales including or consisting of a presence of hypertension scale, a presence of smoking behavior scale, a serum level of soluble ST2 scale, a serum level of N-terminal pro-brain natriuretic peptide (NT-proBNP) scale, an age of the subject scale, a body mass index scale, and a presence of diabetes scale; (b) a point scale; and (c) a predictor scale. An example of one such nomogram is shown in FIG. 23.

In some embodiments, each of the nomograms provided herein is designed such that each of the plurality of scales listed in (a) has values, the plurality of scales listed in (a) is depicted on the two-dimensional or three-dimensional support with respect to the point scale in (b), such that the values on each of the plurality of scales can be correlated with values on the point scale, and the predictor scale contains information correlating a sum of each of correlated values on the point scale to the quantitative probability that a subject not diagnosed or presenting with heart failure will develop heart failure within the specific time period.

In some embodiments, the subject has further not been previously identified as being at risk of developing a disease (e.g., any cardiovascular disease, pulmonary disease, renal insufficiency, stroke, or any of the ST2 related diseases described herein). In some embodiments, the subject has further not been diagnosed as having a disease (e.g., any cardiovascular disease, pulmonary disease, renal insufficiency, stroke, or any of the ST2 related diseases described herein) and/or does not present with one or more symptoms of a disease (e.g., any cardiovascular disease, pulmonary disease, renal insufficiency, stroke, or any of the ST-2 related diseases described herein). Non-limiting examples of ST2-related diseases include, without limitation, cardiovascular disease, pulmonary disease, sepsis, Kawasaki disease, and Th2-associated diseases. In some embodiments, the subject presents with one or more non-specific symptoms that include, but are not limited to, chest pain or discomfort, shortness of breath, nausea, vomiting, eructation, sweating, palpitations, lightheadedness, fatigue, and fainting. In some embodiments, the subject has previously been identified as being at risk of developing heart failure. In some embodiments, the subject further has hypertriglyceridemia and/or hypercholesterolemia.

In any of the nomograms described herein, the two-dimensional support can be, e.g., a card, a piece of paper or cardboard, or a visual screen or display (e.g., a display on a hand-held device). Any of the nomograms described herein can be designed as shown in the exemplary nomograms in the Examples. As can be appreciated by those skilled in the art, the nomograms can be designed in several different ways. Non-limiting examples of designs that can be used for the presently provided nomograms are described in U.S. Pat. Nos. 6,409,664 and 5,993,388.

In any of the nomograms provided herein, the time period is between about 1 year and about 10 years (e.g., between about 1 year and 9 years, between about 1 year and 8 years, between about 1 year and 7 years, between about 1 year and 6 years, between about 1 year and 5 years, between about 1 year and 4 years, between about 1 year and 3 years, between about 1 year and 2 years, between about 2 years and 10 years, between about 2 years and 9 years, between about 2 years and 8 years, between about 2 years and 7 years, between about 2 years and 6 years, between about 2 years and 5 years, between about 2 years and 4 years, between about 3 years and 10 years, between about 3 years and 9 years, between about 3 years and 8 years, between about 3 years and 7 years, between about 3 years and 6 years, between about 3 years and 5 years, between about 4 years and 10 years, between about 4 years and 9 years, between about 4 years and 8 years, between about 4 years and 7 years, between about 4 years and 6 years, between about 5 years and about 10 years, between about 5 years and about 9 years, between about 5 years and about 8 years, between about 5 years and about 7 years, between about 6 years and about 10 years, between about 6 years and about 9 years, between about 6 years and about 8 years, between about 7 years and about 10 years, between about 7 years and 9 years, or between about 8 years and about 10 years). In some embodiments of the nomograms, the period of time is 1 year, 18 months, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years.

Also provided are methods of determining the quantitative probability that a subject not diagnosed or presenting with heart failure will develop heart failure within a specific time period comprising the use of any of the nomograms described herein.

### Methods of Determining the Risk of Developing Heart Failure

Also provided are methods of determining the risk of developing heart failure within a specific time period in a subject not diagnosed or presenting with heart failure that include: (a) providing a set of factors relating to the subject's health including or consisting of one or more (e.g., two, three, four, five, six, seven, or eight) of: presence or absence of hypertension in the subject, presence or absence of coronary artery disease in the subject, smoking or non-smoking behavior of the subject, body mass index of the subject, serum level of soluble ST2 in the subject, serum level of N-terminal pro-brain natriuretic peptide (NT-proBNP) in the subject, age of the subject, and presence or absence of diabetes in the subject; (b) determining a separate point value for each of the provided factors in (a); (c) adding the separate point values for each of the provided factors in (b) together to yield a total points value; and (d) determining the subject's risk of developing heart failure within a specific time period by correlating the total points value in (c) with a value on a predictor scale of risk of developing heart failure within the specific time period based on the set of factors obtained from a population of subjects not diagnosed or presenting with heart failure.

In some embodiments, the set of factors includes or consists of: presence or absence of hypertension in the subject, smoking or non-smoking behavior of the subject, serum level of soluble ST2 in the subject, age of the subject, body mass index of the subject, and presence or absence of diabetes in the subject. In some embodiments, the set of factors includes or consists of: presence or absence of hypertension in the subject, presence or absence of coronary artery disease in the subject, smoking or non-smoking behavior of the subject, serum level of soluble ST2 in the subject, age of the subject, body mass index of the subject, and presence or absence of diabetes in the subject. In other embodiments, the set of factors includes or consists of presence or absence of hypertension in the subject, presence or absence of coronary artery disease in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject. In some embodiments, the set of factors includes or consists of presence or absence of hypertension in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject.

In some embodiments, the predictor scale can be based on the set of factors obtained from a population of subjects further self-identified as healthy. In some embodiments, the predictor scale can be based on the set of factors obtain from a population of subjects not previously identified as being at risk of developing a disease (e.g., any cardiovascular disease, pulmonary disease, renal insufficiency, stroke, or any of the ST-2 related diseases described herein), not diagnosed as having a disease (e.g., any cardiovascular disease, pulmonary disease, renal insufficiency, stroke, or any of the ST-2 related diseases described herein), and/or not presenting with one or more symptoms of a disease (e.g., any cardiovascular disease, pulmonary disease, renal insufficiency, stroke, or any of the ST-2 related diseases described herein).

In some embodiments, the subject has further not been previously identified as being at risk of developing a disease (e.g., any cardiovascular disease, pulmonary disease, renal insufficiency, stroke, or any of the ST-2 related diseases described herein). In some embodiments, the subject has further not been diagnosed as having a disease (e.g., any cardiovascular disease, pulmonary disease, renal insufficiency, stroke, or any of the ST2-related diseases described herein) and/or does not present with one or more symptoms of a disease (e.g., any cardiovascular disease, pulmonary disease, renal insufficiency, stroke, or any of the ST2-related diseases described herein). Non-limiting examples of ST2-related diseases include, without limitation, cardiovascular disease, pulmonary disease, sepsis, Kawasaki disease, and Th2-associated diseases. In some embodiments, the subject presents with one or more non-specific symptoms that include, but are not limited to, chest pain or discomfort, shortness of breath, nausea, vomiting, eructation, sweating, palpitations, lightheadedness, fatigue, and fainting. In some embodiments, the subject has previously been identified as being at risk of developing heart failure. In some embodiments, the subject further has hypertriglyceridemia and/or hypercholesterolemia.

In some embodiments of the methods described herein, the providing in (a) includes obtaining the set of factors from the subject's recorded clinical information. In some embodiments of the methods described herein, the obtaining is performed through a computer software program. In some examples, the providing in (a) includes the manual entry of the set of factors into a website interface or a software program. For example, the manual entry can be performed by the subject or can be performed by a health care professional. Additional examples of how any of the factors can be provided are described herein. Any of the methods for providing any of the factors described herein can be used in these methods in any combination (without limitation).

In any of the methods described herein, the time period is between about 1 year and about 10 years (e.g., between about 1 year and 9 years, between about 1 year and 8 years, between about 1 year and 7 years, between about 1 year and 6 years, between about 1 year and 5 years, between about 1 year and 4 years, between about 1 year and 3 years, between about 1 year and 2 years, between about 2 years and 10 years, between about 2 years and 9 years, between about 2 years and 8 years, between about 2 years and 7 years, between about 2 years and 6 years, between about 2 years and 5 years, between about 2 years and 4 years, between about 3 years and 10 years, between about 3 years and 9 years, between about 3 years and 8 years, between about 3 years and 7 years, between about 3 years and 6 years, between about 3 years and 5 years, between about 4 years and 10 years, between about 4 years and 9 years, between about 4 years and 8 years, between about 4 years and 7 years, between about 4 years and 6 years, between about 5 years and about 10 years, between about 5 years and about 9 years, between about 5 years and about 8 years, between about 5 years and about 7 years, between about 6 years and about 10 years, between about 6 years and about 9 years, between about 6 years and about 8 years, between about 7 years and about 10 years, between about 7 years and 9 years, or between about 8 years and about 10 years). In some embodiments of any of the methods described herein, the period of time is 1 year, 18 months, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years.

Some embodiments further include determining one or more of the set of factors in (a) in the subject (e.g., using any combination of the methods for providing or determining one or more of presence or absence of hypertension, smoking or non-smoking behavior, serum level of soluble ST2, age, body mass index, presence or absence of diabetes, presence or absence of coronary artery disease, and serum level of NT-proBNP in the subject described herein or known in the art). For example, a serum level of soluble ST2 in a subject can be determined by obtaining a biological sample from the subject (e.g., a biological sample containing serum) and determining the level of soluble ST2 in the sample (e.g., by performing an assay using an antibody that specifically binds to soluble ST2). In some embodiments, the sample contains blood, serum, or plasma. The presence of hypertension in a subject can be, e.g., characterized as one or both of systolic pressure of ≧140 mmHg and diastolic pressure of ≧90 mmHg.

Some embodiments further include recording the subject's determined risk into the subject's medical file or record (e.g., a medical file or record stored in a computer readable medium). Some embodiments further include providing information regarding the subject's determined risk to one or more family members or one or more of the subject's health care providers.

Any of the methods described herein can be performed, e.g., using a nomogram (e.g., any of the exemplary nomograms described herein), or using a computer-based system, e.g., a software program or application (app). In some embodiments, the determining in (b), the adding in (c), and the determining in (d) is performed using a software program.

Some embodiments further include comparing the determined risk of developing heart failure within the specific time period to a predetermined risk value, identifying a subject whose determined risk of developing heart failure within the specific time period is elevated as compared to the predetermined risk value, and administering a treatment for reducing the risk of developing heart failure to the identified subject. In some embodiments of these methods, the comparing in (e) and the identifying in (f) are performed using a software program. Exemplary treatments for reducing the risk of developing heart failure are described herein. For example, the treatment can be selected from the group consisting of: an anti-inflammatory agent, an anti-thrombotic agent, an anti-platelet agent, a fibrinolytic agent, a lipid-reducing agent, a direct thrombin inhibitor, a glycoprotein IIb/IIIa receptor inhibitor, a calcium channel blocker, a beta-adrenergic receptor blocker, a cyclooxygenase-2 inhibitor, and a renin-angiotensin-aldosterone system (RAAS) inhibitor. Non-limiting examples of RAAS inhibitors include an angiotensin-converting enzyme (ACE) inhibitor, an angiotensin II receptor blocker (ARB), aldosterone antagonists, an angiotensin II receptor antagonist, an agent that activates the catabolism of angiotensin II, and an agent that prevents the synthesis of angiotensin I. Non-limiting examples of lipid-reducing agents include gemfibrozil, cholestyramine, colestipol, nicotinic acid, probucol, lovastatin, fluvastatin, simvastatin, atorvastatin, pravastatin, and cerivastatin. Additional examples of treatments for reducing the risk of developing heart failure are exercise therapy, smoking cessation therapy, and nutritional consultation. Additional examples of treatments for reducing the risk of developing heart failure include increased periodicity of clinical evaluation, e.g., clinical evaluation of cardiovascular disease (e.g., cardiac testing).

### Methods of Selecting a Treatment for a Subject

Also provided are methods of selecting a therapeutic treatment for a subject that include determining the subject's risk of developing heart failure within a specific time period (e.g., using any of the methods, nomograms, or computer methods/programs described herein), identifying a subject determined to have an elevated risk of developing heart failure within a specific time period (e.g., as compared to a healthy control subject or a healthy control subject population), and selecting a treatment for reducing the risk of developing heart failure for the subject. Some embodiments further include administering the selected treatment to the subject.

In some embodiments, the subject has further not been previously identified as being at risk of developing a disease (e.g., any cardiovascular disease, pulmonary disease, renal insufficiency, stroke, or any of the ST-2 related diseases described herein). In some embodiments, the subject has further not been diagnosed as having a disease (e.g., any cardiovascular disease, pulmonary disease, renal insufficiency, stroke, or any of the ST-2 related diseases described herein) and/or does not present with one or more symptoms of a disease (e.g., any cardiovascular disease, pulmonary disease, renal insufficiency, stroke, or any of the ST-2 related diseases described herein). Non-limiting examples of ST2-associated conditions include, without limitation, cardiovascular disease, pulmonary disease, sepsis, Kawasaki disease, and Th2-associated diseases. In some embodiments, the subject presents with one or more non-specific symptoms that include, but are not limited to, chest pain or discomfort, shortness of breath, nausea, vomiting, eructation, sweating, palpitations, lightheadedness, fatigue, and fainting. In some embodiments, the subject has previously been identified as being at risk of developing heart failure. In some embodiments, the subject has hypertriglyceridemia and/or hypercholesterolemia.

For example, the treatment for reducing the risk of heart failure can be selected from the group of: nitrates, calcium channel blockers, diuretics, thrombolytic agents, digitalis, renin-angiotensin-aldosterone system (RAAS) modulating agents (e.g., beta-adrenergic blocking agents (e.g., alprenolol, bucindolol, carteolol, carvedilol, labetalol, nadolol, penbutolol, pindolol, propranolol, sotalol, timolol, cebutolol, atenolol, betaxolol, bisoprolol, celiprolol, esmolol, metoprolol, and nebivolol), angiotensin-converting enzyme inhibitors (e.g., benazepril, captopril, enalapril, fosinopril, lisinopril, moexipril, perindopril, quinapril, ramipril, and trandolapril), aldosterone antagonists (e.g., spironolactone, eplerenone, canrenone (canrenoate potassium), prorenone (prorenoate potassium), and mexrenone (mexrenoate potassium)), renin inhibitors (e.g., aliskiren, remikiren, and enalkiren), and angiotensin II receptor blockers (e.g., valsartan, telmisartan, losartan, irbesartan, and olmesartan)), and cholesterol-lowering agents (e.g., a statin). Additional methods for treatment are also known in the art, e.g., Braunwald's Heart Disease: A Textbook of Cardiovascular Medicine, Single Volume, 9th Edition. The selected treatment can also be the administration of at least one or more new therapeutic agents to the subject, an alteration (e.g., increase or decrease) in the frequency, dosage, or length of administration of one or more therapeutic agents to the subject, or the removal of at least one or more therapeutic agents from the patient's treatment regime. The selected treatment can also be inpatient care of the subject (e.g., admittance or re-admittance of the subject to a hospital (e.g., an intensive care or critical care unit) or an assisted-care facility). In some embodiments, the selected treatment is surgery (e.g., organ or tissue transplant or angioplasty). In some embodiments, the selected treatment can include increased cardiac monitoring in the subject. In examples, the selected treatment can include cardiac assessment using one or more of the following techniques: electrocardiogram, wearing an event monitor, cardiac stress testing, echocardiography, cardiovascular magnetic resonance imaging, ventriculography, cardiac catheterization, coronary catheterization, cardiac positron emission tomography, cardiac computed tomography, angiocardiography, and electrophysiology study. In some embodiments, the selected treatment is aggressive medical treatment that can include, e.g., inpatient treatment (e.g., in a hospital, acute or critical care department, or an assisted-care facility). In another example, aggressive medical treatment includes increased periodicity of clinical evaluation, e.g., clinical evaluation of cardiovascular disease (e.g., cardiac testing). In some embodiments, the selected treatment can be exercise therapy, smoking cessation therapy, and nutritional consultation.

### Methods of Determining the Efficacy of Treatment

Also provided herein are methods for determining the efficacy of a treatment for reducing the risk of developing heart failure in a subject. These methods can include all or some of: (a) providing a set of factors relating to the subject's health (e.g., any of the sets of factors described herein) at a first time point; (b) determining a separate point value for each of the provided factors in (a); (c) adding the separate point values for each of the provided factors in (b) together to yield a total points value; (d) determining the subject's risk of developing heart failure within a specific time period at the first time point by correlating the total points value of (c) with a value on a predictor scale of risk of developing heart failure within the specific time period based on the set of factors obtained from a population of subjects not diagnosed or presenting with heart failure; (e) providing a set of factors (e.g., any of the sets of factors described herein or the same set of factors as in (a)) relating to the subject's health at a second time point; (f) determining a separate point value for each of the provided factors in (e); (g) adding the separate point values for each of the provided factors in (f) together to yield a total points value; (h) determining the subject's risk of developing heart failure within the specific time period at the second time point by correlating the total points value of (g) with a value on a predictor scale of risk of developing heart failure within the specific time period based on the set of factors obtained from a population of subjects not diagnosed or presenting with heart failure, where the second time point is after the first time point, and the subject has received a treatment (e.g., at least two doses of a treatment) after the first time point and before the second time point; (i) comparing the subject's risk of developing heart failure within the specific time period determined at the second time point to the subject's risk of developing heart failure within the specific time period determined at the first time point; and/or (j) identifying the treatment administered to a subject having a decreased risk of developing heart failure within the specific time period determined at the second time point as compared the subject's risk of developing heart failure within the specific time period determined at the first time point as being effective for reducing the risk of developing heart failure, or identifying the treatment administered to a subject having an elevated risk of developing heart failure within the specific time period determined at the second time point as compared to the subject's risk of developing heart failure within the specific time period determined at the first time point as not being effective for reducing the risk of developing heart failure.

In some embodiments, the predictor scale can be based on the set of factors obtained from a population of subjects further self-identified as healthy. In some embodiments, the predictor scale can be based on the set of factors obtained from a population of subjects not previously identified as being at risk of developing a disease (e.g., any cardiovascular disease, pulmonary disease, renal insufficiency, stroke, or any of the ST2-related diseases described herein), not diagnosed as having a disease (e.g., any cardiovascular disease, pulmonary disease, renal insufficiency, stroke, or any of the ST2-related diseases described herein), and/or not presenting with one or more symptoms of a disease (e.g., any cardiovascular disease, pulmonary disease, renal insufficiency, stroke, or any of the ST2-related diseases described herein).

In some embodiments, the subject has further not been previously identified as being at risk of developing a disease (e.g., any cardiovascular disease, pulmonary disease, renal insufficiency, stroke, or any of the ST-2 related diseases described herein). In some embodiments, the subject has further not been diagnosed as having a disease (e.g., any cardiovascular disease, pulmonary disease, renal insufficiency, stroke, or any of the ST-2 related diseases described herein) and/or does not present with one or more symptoms of a disease (e.g., any cardiovascular disease, pulmonary disease, renal insufficiency, stroke, or any of the ST-2 related diseases described herein). Non-limiting examples of ST2-associated conditions include, without limitation, cardiovascular disease, pulmonary disease, sepsis, Kawasaki disease, and Th2-associated diseases. In some embodiments, the subject presents with one or more non-specific symptoms that include, but are not limited to, chest pain or discomfort, shortness of breath, nausea, vomiting, eructation, sweating, palpitations, lightheadedness, fatigue, and fainting. In some embodiments, the subject has previously been identified as being at risk of developing heart failure. In some embodiments, the subject further has hypertriglyceridemia and/or hypercholesterolemia. In some embodiments, the subject has been previously treated with an agent for reducing the risk of developing heart failure. In other examples, the subject has previously been administered a treatment for reducing the risk of heart failure, and the previous treatment was determined to be ineffective in the subject.

In some embodiments, the set of factors in (a) and/or (e) includes or consists of presence or absence of hypertension in the subject, smoking or non-smoking behavior of the subject, serum level of soluble ST2 in the subject, age of the subject, body mass index of the subject, and presence or absence of diabetes in the subject. In some embodiments, the set of factors in (a) and/or (e) includes or consists of presence or absence of hypertension in the subject, presence or absence of coronary artery disease in the subject, smoking or non-smoking behavior of the subject, serum level of soluble ST2 in the subject, age of the subject, body mass index of the subject, and presence or absence of diabetes in the subject. In other embodiments, the set of factors in (a) and/or (e) includes or consists of presence or absence of hypertension in the subject, presence or absence of coronary artery disease in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject. In some embodiments, the set of factors in (a) and/or (e) includes or consists of presence or absence of hypertension in the subject, smoking or non-smoking behavior of the subject, serum level of soluble ST2 in the subject, serum level of N-terminal pro-brain natriuretic peptide (NT-proBNP) in the subject, age of the subject, body mass index of the subject, and presence or absence of diabetes in the subject.

In any of the methods described herein, the time period is between about 1 year and about 10 years (e.g., between about 1 year and 9 years, between about 1 year and 8 years, between about 1 year and 7 years, between about 1 year and 6 years, between about 1 year and 5 years, between about 1 year and 4 years, between about 1 year and 3 years, between about 1 year and 2 years, between about 2 years and 10 years, between about 2 years and 9 years, between about 2 years and 8 years, between about 2 years and 7 years, between about 2 years and 6 years, between about 2 years and 5 years, between about 2 years and 4 years, between about 3 years and 10 years, between about 3 years and 9 years, between about 3 years and 8 years, between about 3 years and 7 years, between about 3 years and 6 years, between about 3 years and 5 years, between about 4 years and 10 years, between about 4 years and 9 years, between about 4 years and 8 years, between about 4 years and 7 years, between about 4 years and 6 years, between about 5 years and about 10 years, between about 5 years and about 9 years, between about 5 years and about 8 years, between about 5 years and about 7 years, between about 6 years and about 10 years, between about 6 years and about 9 years, between about 6 years and about 8 years, between about 7 years and about 10 years, between about 7 years and 9 years, or between about 8 years and about 10 years). In some embodiments of the methods described herein, the period of time is 1 year, 18 months, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years.

In some examples, the time difference between the first and second time periods is at least one week, at least two weeks, at least 1 months, at least 2 months, at least 3 months, at least 4 months, at least 5 months, at least 6 months, at least 7 months, at least 8 months, at least 9 months, at least 10 months, at least 11 months, or at least 12 months. In some embodiments that subject is administered at least three doses, at least four doses, at least five doses, at least 6 doses, at least 7 doses, at least 8 doses, at least 9 doses, at least 10 doses, at least 12 doses, at least 14 doses, at least 16 doses, at least 18 doses, at least 20 doses, at least 25 doses, at least 30 doses, at least 40 doses, at least 50 doses, at least 60 doses, at least 70 doses, at least 80 doses, at least 90 doses, or at least 100 doses of the treatment between the first time point and the second time point.

In some embodiments of any of these methods, one or both of the providing in (a) and the providing in (e) includes obtaining the set of factors from a subject's recorded clinical information (e.g., the subject's clinical file). For example, the obtaining can be performed through a computer software program. One or both of the providing in (a) and the providing in (e) can include the manual entry of the set of factors into a website interface. For example, the manual entry can be performed by the subject or a health care professional.

In some embodiments, the providing of the one or more factors includes determining the one or more of the set of factors at one or both of the first and second time points. Non-limiting examples of how to determine and provide each factor in the set of factors in a subject are described herein. Additional examples of how to determine or provide each factor in the set of factors are known in the art. In some embodiments, the presence of hypertension in a subject is characterized as one or both of systolic pressure of ≧140 mm Hg and diastolic pressure of ≧90 mm Hg.

Some embodiments further include recording the determined efficacy of the treatment into the subject's medical file or record. In some embodiments, the subject's medical file or record is stored in a computer readable medium, and, optionally, the computer readable medium is modified to include information regarding the determined efficacy of the treatment in the subject. In some embodiments, the determining in one or both of steps (b) and (d) and/or the determining in one or both of steps (f) and (h) is performed using a nomogram (e.g., any of the nomograms described herein). In some embodiments, one or more of the determining in (b), the adding in (c), and the determining in (d) is performed using a software program and/or one or more of the determining in (f), the adding in (g), and the determining in (h) is performed using a software program. In some embodiments, one or both of the comparing in (i) and the identifying in (j) are performed using a software program.

Some embodiments further include administering the treatment for reducing the risk of developing heart failure (e.g., at least two doses of the treatment for reducing the risk of developing heart failure) to the identified subject after the first time point and before the second time point. In some embodiments, the treatment is administration of an agent selected from the group of: an anti-inflammatory agent, an anti-thrombotic agent, an anti-platelet agent, a fibrinolytic agent, a lipid-reducing agent, a direct thrombin inhibitor, a glycoprotein IIb/IIIa receptor inhibitor, a calcium channel blocker, a beta-adrenergic receptor blocker, a cyclooxygenase-2 inhibitor, and a renin-angiotensin-aldosterone system (RAAS) inhibitor. For example, a RAAS inhibitor can be any of: an angiotensin-converting enzyme (ACE) inhibitor, an angiotensin II receptor blocker (ARB), aldosterone antagonists, an angiotensin II receptor antagonist, an agent that activates the catabolism of angiotensin II, and an agent that prevents the synthesis of angiotensin I. Non-limiting examples of lipid-reducing agents are gemfibrozil, cholestyramine, colestipol, nicotinic acid, probucol, lovastatin, fluvastatin, simvastatin, atorvastatin, pravastatin, and cerivastatin. The treatment can also be exercise therapy, smoking cessation therapy, and nutritional consultation. Additional examples of treatments for reducing the risk of developing heart failure described herein and known in the art can be administered to the subject after the first time point and before the second time point.

In some embodiments, where the treatment administered is found to be effective, the subject is administered the same treatment. In some embodiments, where the treatment administered is found to be ineffective, the subject is administered a different treatment (e.g., a different treatment for reducing the risk of developing heart failure, e.g., any of the treatments described herein) or a different dose (e.g., a higher dose or more frequent dosing) of the same treatment (for pharmacological treatments).

### Methods of Selecting a Subject for Participation in a Clinical Trial

Also provided herein are methods of selecting a subject for participation in a clinical trial (e.g., a clinical trial of a treatment for reducing the risk of developing heart failure in a subject). These methods can include determining the subject's risk of developing heart failure using any of the methods, nomograms, or computer systems/programs described herein, identifying a subject as having an elevated risk of developing heart failure within a specific time period (e.g., as compared to a healthy control subject or a healthy control subject population), and selecting the subject for participation in a clinical study (e.g., a clinical study to test a candidate treatment for reducing the risk of developing heart failure). Some embodiments further include a step of administering to the selected subject a candidate treatment for reducing the risk of developing heart failure. Any of the subjects described herein can be selected for participation in a clinical trial (e.g., a clinical trial of a candidate treatment for reducing the risk of heart failure). In some embodiments, a subject determined not to have an elevated risk of developing heart failure is not selected for participation in a clinical trial or is selected as a control population in a clinical trial.

### Systems

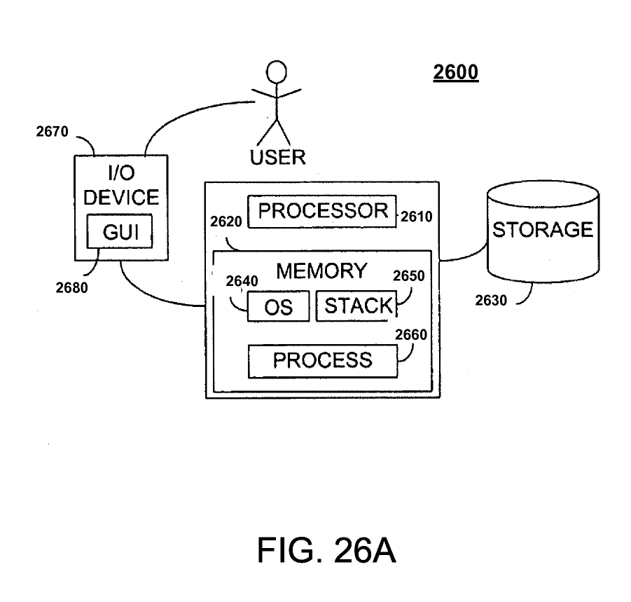

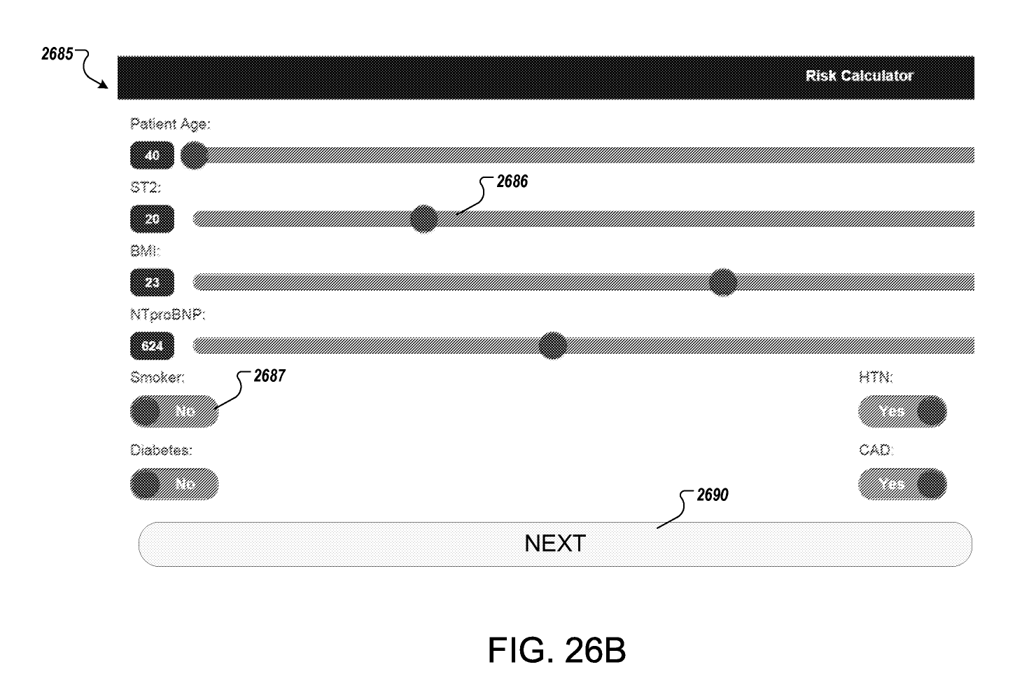

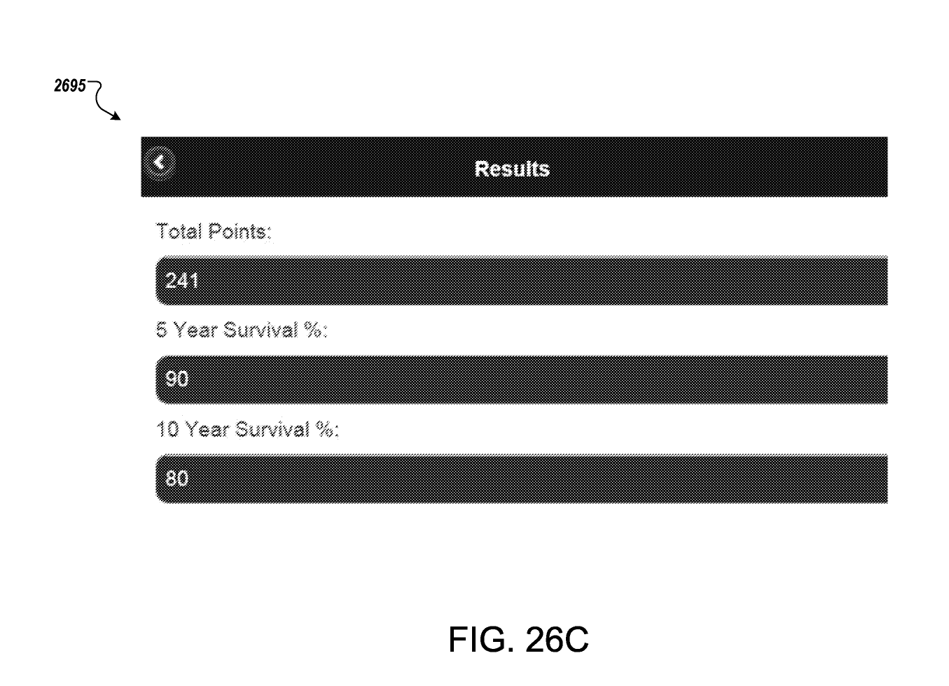

Any of the methods and nomograms described herein can be implemented in a system 2600 as shown in FIG. 26A; other systems and devices as known in the art can also be used. In some implementations, the system 2600 can be embodied in a desktop or laptop computer, or a mobile device such as a cellular phone, tablet device, or e-reader. The exemplary system 2600 includes a processor 2610, a memory 2620, and a storage device 2630; in some embodiments, the system does not include one or both of memory and/or a storage device. The memory 2620 includes an operating system (OS) 2640, such as Linux, UNIX, or Windows® XP, a TCP/IP stack 2650 for communicating with a network (not shown), and a process 2660 for analyzing data in accordance with the technology described in this document. In some implementations, the system 2600 also includes a link to an input/output (I/O) device 2670 for display of a graphical user interface (GUI) 2680 to a user.

In some implementations, the GUI 2680 can include an input interface. An example of an input interface 2685 is shown in FIG. 26B. The input interface 2685 can allow a user to manually enter one or more of the set of factors used in the risk calculation. In the example shown in FIG. 26B, the input interface 2685 allows the user to enter, for example, the user's age, level of ST2, BMI, and level of NT-proBNP using adjustable slider scales 2686. The input interface 2685 also includes user selectable graphical switches 2687 that allows the user to enter binary information such as whether or not the user is a smoker, and whether or not the user has diabetes. Other forms of input, such as data entry fields, or selectable buttons can also be used on the input interface 2685. In some implementations, the input interface can include a control, which upon activation, can allow for data to be imported from a remote data source. For example, the input interface 2685 may include a control that enables a user to allow access to a remote database from which one or more of the set of factors can be imported. The input interface can also include a control 2690 that causes a risk calculation based on the factors entered using the input interface 2685.

In some implementations, activation of the control 2690 can cause a display of an output interface. An example of such an output interface 2695 is shown in FIG. 26C. The output interface 2695 can include, for example, a display of the total points calculated from the set of factors, a probability of 5-year heart failure-free survival, and a probability of 10-year heart failure-free survival. The output interface can include, for example, a display of the total points calculated from the set of factors, a risk of developing heart failure within a time period of 5 years, and a risk of developing heart failure within a time period of 10 years. The output interface 2695 can also include, for example, graphical representations related to the risk calculation. In some implementations, the graphical representations in the output interface 2695 can be made interactive.

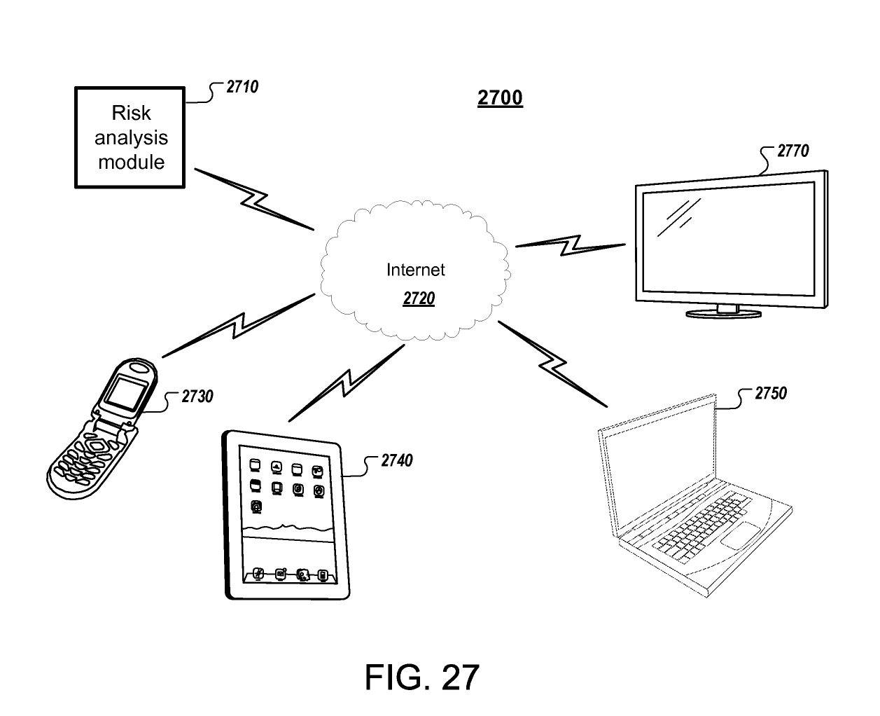

In some implementations, the risk analysis functionalities described herein may also be implemented within a network environment. An example of such a network environment 2700 is shown in FIG. 27. As shown in the example of FIG. 27, the networking environment 2700 provides users (e.g., individuals such as clinicians, nurses, physician assistants, clinical laboratory workers, patients, or family members of patients) access to information collected, produced, and/or stored by a risk analysis module 2710. For example, the risk analysis module may be an entity (or multiple entities) that employs one or more computing devices (e.g., servers, computer systems, etc.) to process information related to the set of factors. The risk analysis module can include a system 2600 as described with reference to FIG. 26. In some implementations, the risk analysis module 2710 may execute one or more processes for determining a subject's risk of developing heart failure within a period of time, in accordance with any of the methods described in this document.

Various techniques and methodologies may be implemented for exchanging information between the users and the risk analysis module 2710. For example, one or more networks (e.g., the Internet 2720) may be employed for interchanging information with user devices. As illustrated in FIG. 27, various types of computing devices and display devices may be employed for information exchange. For example, hand-held computing devices (e.g., a cellular telephone 2730, tablet computing device 2740, etc.) may exchange information through one or more networks (e.g., the Internet 2720) with the risk analysis module 2710. Other types of computing devices such as a laptop computer 2750 and other computer systems may also be used to exchange information with the risk analysis module 2710. A display device such as a liquid crystal display (LCD) television 2770 or other display device may also present information from the risk analysis module 2710. One or more types of information protocols (e.g., file transfer protocols, etc.) may be implemented exchanging information. The user devices may also present one or more types of interfaces (e.g., the input or output user interfaces) to exchange information between the user and the risk analysis module 2710. For example, a network browser may be executed by a user device to establish a connection with a website (or webpage) of the risk analysis module 2710 and provide a vehicle for exchanging information. The risk analysis module 2710 can include software and hardware configured to perform the risk calculations from the set of factors in accordance with the description provided in this document.

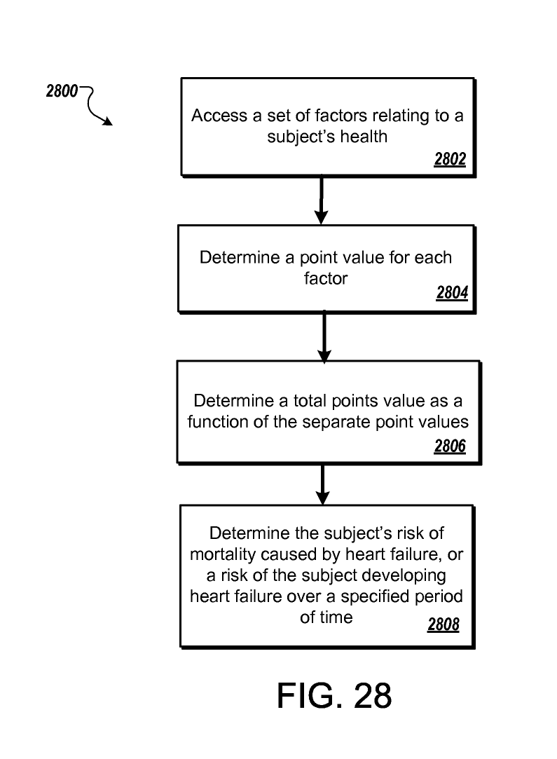

FIG. 28 depicts a flowchart 2800 illustrating an example sequence of operations for determining a subject's risk of developing heart failure within a specified period of time. The operations depicted in the flowchart 2800 can be performed, for example, by a processor 2600 or a risk analysis module 2710 described with reference to FIGS. 26A and 27, respectively. The operations can include accessing a set of factors related to subject's health (2802). The set of factors can include, for example, one or more of: a presence or absence of hypertension in the subject, smoking or non-smoking behavior of the subject, a presence or absence of coronary artery disease in the subject, serum level of soluble ST2 in the subject, serum level of N-terminal pro-brain natriuretic peptide (NT-proBNP) in the subject, age of the subject, body mass index of the subject, and a presence or absence of diabetes in the subject. The set of factors can be accessed from various sources, including, for example, from a database storing the subject's recorded clinical information. Accessing the set of factors can also include receiving one or more of the factors via a user interface, such as, e.g., the input interface described above with reference to FIG. 26B.

Operations can also include determining a point value for each of the factors (2804). The point value for each of the factors can be determined based on one or more scales that relate the factors to a numerical value. For example, each of the following factors can be assigned a numerical value: presence or absence of hypertension in the subject, presence or absence of coronary artery disease in the subject, smoking or non-smoking behavior of the subject, body mass index of the subject, serum level of soluble ST2 in the subject, serum level of N-terminal pro-brain natriuretic peptide (NT-proBNP) in the subject, age of the subject, and presence or absence of diabetes in the subject.

The operations can also include determining total points as a function of the separate point values (2806). In some implementations, the total points can be a sum of the individual point values. In some implementations, the total point can be a more complex function such as a weighted sum, wherein the weight of a particular point value depends on the corresponding factor.

The operations further include determining the subject's risk of developing heart failure within a specified period of time (2808). The risks can be determined, for example, by correlating the total point value with a value on a predictor scale. The predictor scale can be based on a set of factors obtained from a population of subjects not diagnosed or presenting with heart failure. The determined risk can be presented to a user via a user interface such as the output interface described with reference to FIG. 26C. The determined risk can also be stored on a computer readable storage device, for example, as a part of the subject's medical records. The determined risks can also be compared to a predetermined threshold, and an output indicative of the comparison can be provided to a user. For example, if the calculated risk is determined to be above a threshold value, the user may be notified, for example, via a user interface, to contact a health care provider and/or take some actions to mitigate the risk. In some embodiments, the user can be a health care provider (e.g., a clinician) and the health care provider is notified that the subject should be administered a treatment to reduce the risk of developing heart failure (e.g., any of the exemplary treatments for reducing the risk of heart failure described herein or known in the art). In some embodiments, where the user is a health care provider (e.g., a physician) and the health care provider is notified that the treatment administered to the subject is effective for reducing the subject's risk of developing heart failure or ineffective for reducing the subject's risk of developing heart failure (e.g., according to any of the methods described herein).

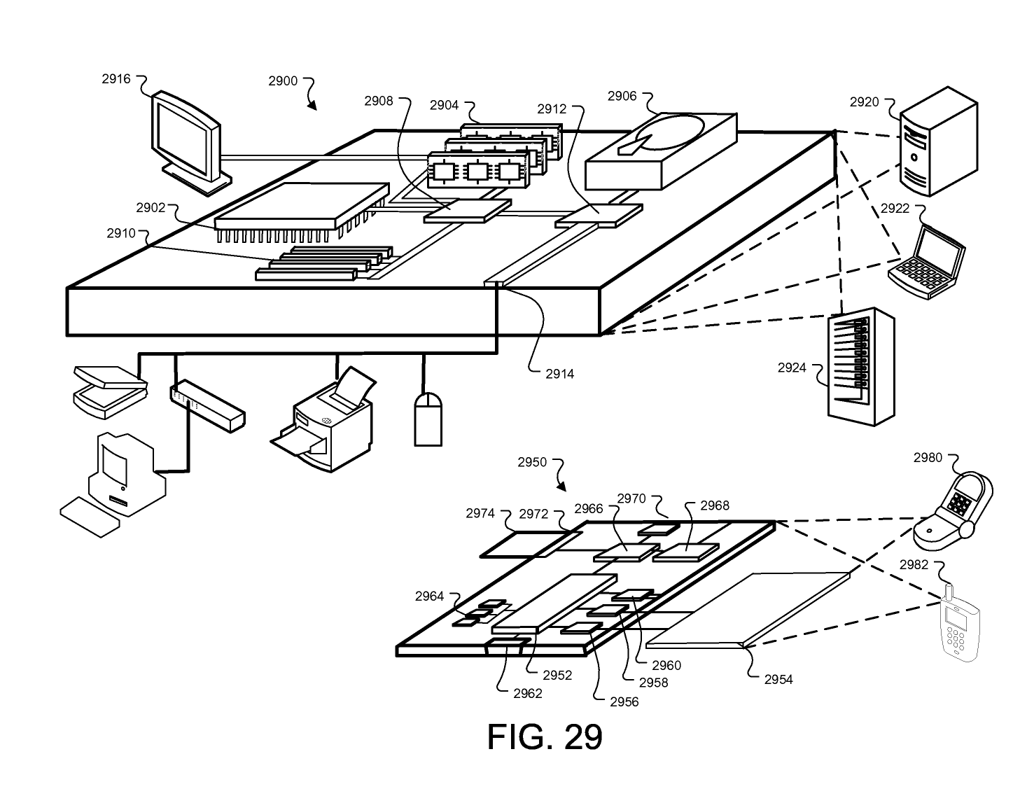

FIG. 29 shows an example of example computer device 2900 and example mobile computer device 2950 that can be used to implement the techniques described herein. For example, a portion or all of the operations of the risk analysis module 2710 may be executed by the computer device 2900 and/or by the mobile computer device 2950 (that may be operated by an end user). Computing device 2900 is intended to represent various forms of digital computers, including, e.g., laptops, desktops, workstations, personal digital assistants, servers, blade servers, mainframes, and other appropriate computers. Computing device 2950 is intended to represent various forms of mobile devices, including, e.g., personal digital assistants, cellular telephones, smartphones, and other similar computing devices. The components shown here, their connections and relationships, and their functions, are meant to be examples, and are not meant to limit implementations of the techniques described and/or claimed in this document.

Computing device 2900 includes a processor 2902, a memory 2904, a storage device 2906, a high-speed interface 2908 connecting to memory 2904 and high-speed expansion ports 2910, and a low speed interface 2912 connecting to a low speed bus 2914 and a storage device 2906. Each of components 2902, 2904, 2906, 2908, 2910, and 2912, are interconnected using various busses, and can be mounted on a common motherboard or in other manners as appropriate. Processor 2902 can process instructions for execution within computing device 2900, including instructions stored in memory 2904 or on storage device 2906 to display graphical data for a GUI on an external input/output device, including, e.g., display 2916 coupled to high speed interface 2908. In other implementations, multiple processors and/or multiple buses can be used, as appropriate, along with multiple memories and types of memory. Also, multiple computing devices 2900 can be connected, with each device providing portions of the necessary operations (e.g., as a server bank, a group of blade servers, or a multi-processor system).

Memory 2904 stores data within computing device 2900. In one implementation, memory 2904 is a volatile memory unit or units. In another implementation, memory 2904 is a non-volatile memory unit or units. Memory 2904 also can be another form of non-transitory computer-readable medium, including, e.g., a magnetic or optical disk.

Storage device 2906 is capable of providing mass storage for computing device 2900. In one implementation, storage device 2906 can be or contain a non-transitory computer-readable medium, including, e.g., a floppy disk device, a hard disk device, an optical disk device, or a tape device, a flash memory, or other similar solid state memory device, or an array of devices, including devices in a storage area network or other configurations. A computer program product can be tangibly embodied in a data carrier. The computer program product also can contain instructions that, when executed, perform one or more methods, including, e.g., those described above. The data carrier is a computer- or machine-readable medium, including, e.g., memory 2904, storage device 2906, memory on processor 2902, and the like.

High-speed controller 2908 manages bandwidth-intensive operations for computing device 2900, while low speed controller 2912 manages lower bandwidth-intensive operations. Such allocation of functions is an example only. In one implementation, high-speed controller 2908 is coupled to memory 2904, display 2916 (e.g., through a graphics processor or accelerator), and to high-speed expansion ports 2910, which can accept various expansion cards (not shown). In the implementation, low-speed controller 2912 is coupled to storage device 2906 and low-speed expansion port 2914. The low-speed expansion port, which can include various communication ports (e.g., USB, Bluetooth®, Ethernet, wireless Ethernet), can be coupled to one or more input/output devices, including, e.g., a keyboard, a pointing device, a scanner, or a networking device including, e.g., a switch or router, e.g., through a network adapter.

Computing device 2900 can be implemented in a number of different forms, as shown in the figure. For example, it can be implemented as standard server 2920, or multiple times in a group of such servers. It also can be implemented as part of a personal computer including, e.g., laptop computer 2922. In some examples, components from computing device 2900 can be combined with other components in a mobile device (not shown), including, e.g., device 2950. Each of such devices can contain one or more of computing device 2900, 2950, and an entire system can be made up of multiple computing devices 2900, 2950 communicating with each other.

Computing device 2950 includes processor 2952, memory 2964, an input/output device including, e.g., display 2954, communication interface 2966, and transceiver 2968, among other components. Device 2950 also can be provided with a storage device, including, e.g., a microdrive or other device, to provide additional storage. Each of components 2950, 2952, 2964, 2954, 2966, and 2968 are interconnected using various buses, and several of the components can be mounted on a common motherboard or in other manners as appropriate.

Processor 2952 can execute instructions within computing device 2950, including instructions stored in memory 2964. The processor can be implemented as a chipset of chips that include separate and multiple analog and digital processors. The processor can provide, for example, for coordination of the other components of device 2950, including, e.g., control of user interfaces, applications run by device 2950, and wireless communication by device 2950.

Processor 2952 can communicate with a user through control interface 2958 and display interface 2956 coupled to display 2954. Display 2954 can be, for example, a TFT LCD (Thin-Film-Transistor Liquid Crystal Display) or an OLED (Organic Light Emitting Diode) display, or other appropriate display technology. Display interface 2956 can comprise appropriate circuitry for driving display 2954 to present graphical and other data to a user. Control interface 2958 can receive commands from a user and convert them for submission to processor 2952. In addition, external interface 2962 can communicate with processor 2942, so as to enable near area communication of device 2950 with other devices. External interface 2962 can provide, for example, for wired communication in some implementations, or for wireless communication in other implementations, and multiple interfaces also can be used.

Memory 2964 stores data within computing device 2950. Memory 2964 can be implemented as one or more of a computer-readable medium or media, a volatile memory unit or units, or a non-volatile memory unit or units. Expansion memory 2974 also can be provided and connected to device 2950 through expansion interface 2972, which can include, for example, a SIMM (Single In Line Memory Module) card interface. Such expansion memory 2974 can provide extra storage space for device 2950, or also can store applications or other data for device 2950. Specifically, expansion memory 2974 can include instructions to carry out or supplement the processes described above, and can also include secure data. Thus, for example, expansion memory 2974 can be provided as a security module for device 2950, and can be programmed with instructions that permit secure use of device 2950. In addition, secure applications can be provided through the SIMM cards, along with additional data, including, e.g., placing identifying data on the SIMM card in a non-hackable manner.

The memory can include, for example, flash memory and/or NVRAM memory, as discussed below. In one implementation, a computer program product is tangibly embodied in a data carrier. The computer program product contains instructions that, when executed, perform one or more methods, including, e.g., any of the methods described herein. The data carrier is a computer- or machine-readable medium, including, e.g., memory 2964, expansion memory 2974, and/or memory on processor 2952 that can be received, for example, over transceiver 2968 or external interface 2962.

Device 2950 can communicate wirelessly through the communication interface 2966, which can include digital signal processing circuitry where necessary, or where desired. Communication interface 2966 can provide for communications under various modes or protocols, including, e.g., GSM voice calls, SMS, EMS, or MMS messaging, CDMA, TDMA, PDC, WCDMA, CDMA2000, or GPRS, among others. Such communication can occur, for example, through radiofrequency transceiver 2968. In addition, short-range communication can occur, including, e.g., using a Bluetooth®, WiFi, or other such transceiver (not shown). In addition, GPS (Global Positioning System) receiver module 2970 can provide additional navigation- and location-related wireless data to device 2950, which can be used as appropriate by applications running on device 2950.

Device 2950 also can communicate audibly using audio codec 2960, which can receive spoken data from a user and convert it to usable digital data. Audio codec 2960 can likewise generate audible sound for a user, including, e.g., through a speaker, e.g., in a handset of device 2950. Such sound can include sound from voice telephone calls, can include recorded sound (e.g., voice messages, music files, and the like) and also can include sound generated by applications operating on device 2950.

Computing device 2950 can be implemented in a number of different forms, as shown in the figure. For example, it can be implemented as cellular telephone 2980. It also can be implemented as part of smartphone 2982, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a display device (e.g., a CRT (cathode ray tube) or LCD (liquid crystal display) monitor) for displaying data to the user and a keyboard and a pointing device (e.g., a mouse or a trackball) by which the user can provide input to the computer. Other kinds of devices can be used to provide for interaction with a user as well; for example, feedback provided to the user can be a form of sensory feedback (e.g., visual feedback, auditory feedback, or tactile feedback); and input from the user can be received in a form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a user interface or a Web browser through which a user can interact with an implementation of the systems and techniques described here), or a combination of such back end, middleware, or front end components. The components of the system can be interconnected by a form or medium of digital data communication (e.g., a communication network). Examples of communication networks include: a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

The invention is further described in the following example, which does not limit the scope of the invention described in the claims.

### EXAMPLES

The invention is further described in the following examples, which do not limit the scope of the invention described in the claims.

### Example 1

### Heart Failure Development Nomograms

Four different nomograms for determining a subject's likelihood of heart failure-free survival within a specific time period were generated and include one or more factors selected from the group of: age, BMI, hypertension, diabetes, coronary artery syndrome, smoking, serum level of soluble ST2, and serum level of NT-proBNP.

The factor of obesity (BMI) can be defined as defined in Table 2 below. The factor of hypertension can be defined as systolic pressure ≧140 mmHg and/or diastolic pressure ≧90 mmHg.

TABLE 2
Obesity Assessment Based on BMI

| BMI | Weight Status |
|---|---|
| Below 18.5 | Underweight |
| 18.5-24.9 | Normal |
| 25.0-29.9 | Overweight |
| 30.0 and Above | Obese |

The four nomograms described in this Example allow for clinicians and patients to perform risk stratification on subjects and provides patients to make lifestyle changes and possibly use pharmacotherapy to modify their risk level, and thus reduce the progress of or development of heart failure (based on their determined likelihood of heart failure-free survival within a specific time period). As is well-appreciated in the art, a medical professional can use a nomogram to determine a total risk score for a subject based on the cumulative effect of the subject's one or more risk factors.

The four exemplary nomograms described herein were based on the Olmsted cohort (a dataset of self-reported healthy patients). Four different models of nomograms for assessment of a subject's likelihood of heart failure-free survival within a specific period of time were compared: a seven parameter model (Model 1), a 7 parameter model minus CAD (Model 2), a 7 parameter model plus NT-proBNP (Model 3), and a 7 parameter model minus CAD and plus NT-proBNP (Model 4). The missing data were imputed except for outcomes. One subject was censored on day 0 (i.e., she was removed from the study). A parametric survival model (Weibull distribution) was generated for each of the four nomogram models (Models 1-4). The validation and calibration were estimated using bootstrap statistical analyses on the same data set.

### Results

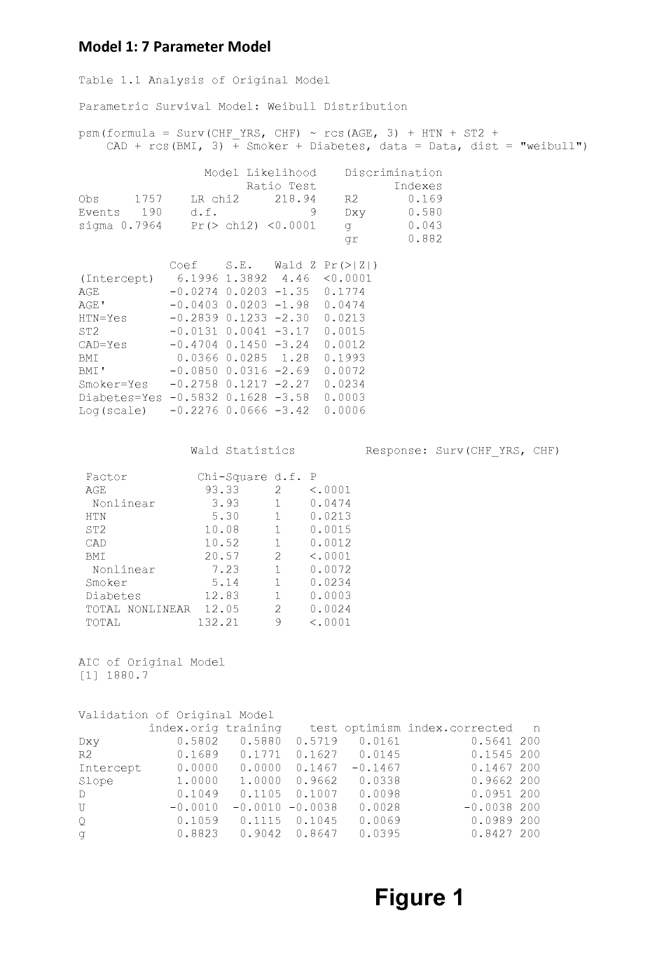

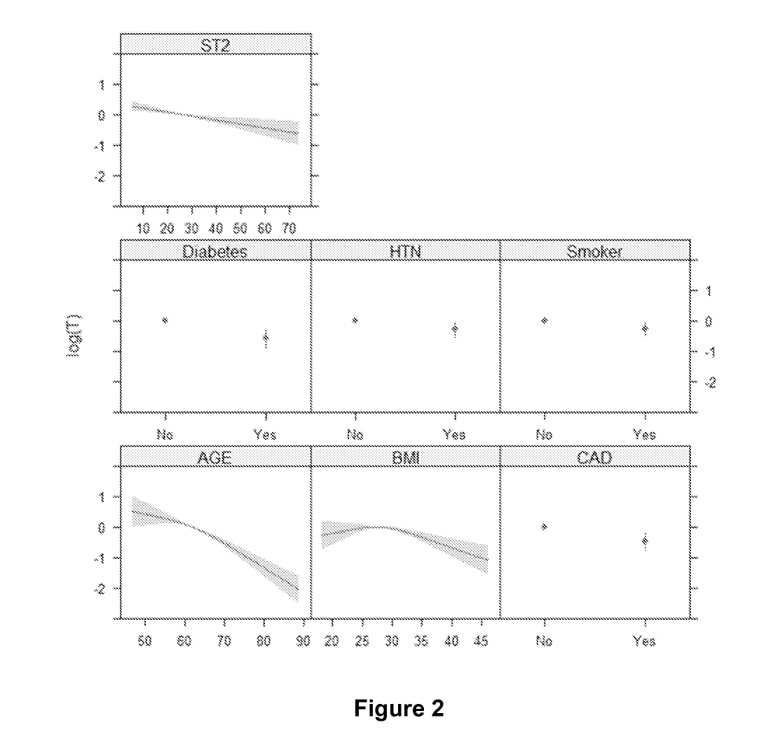

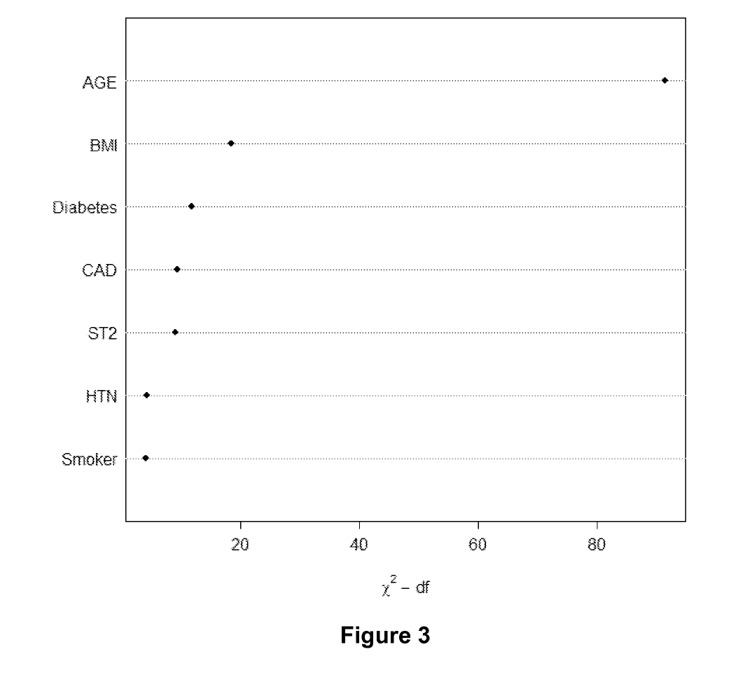

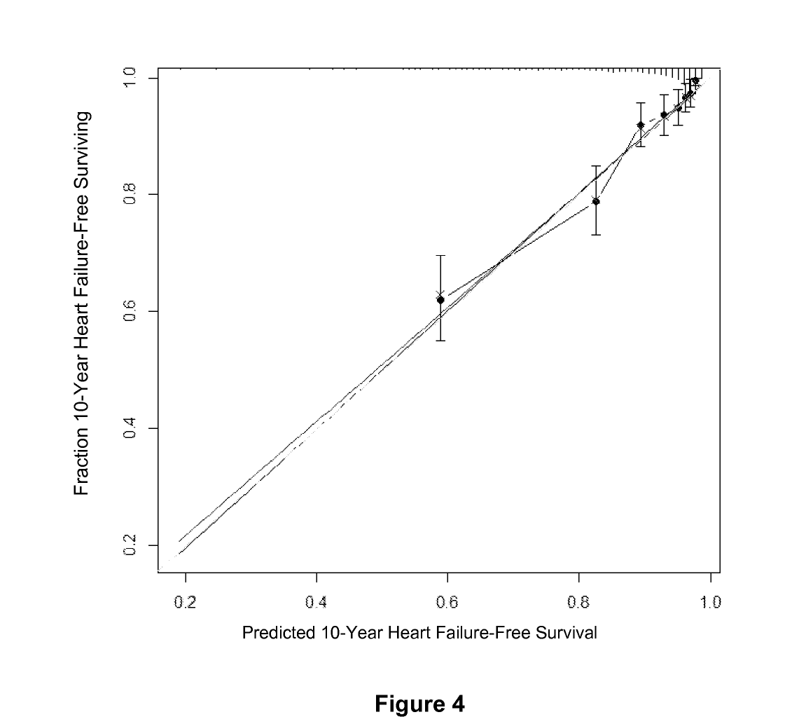

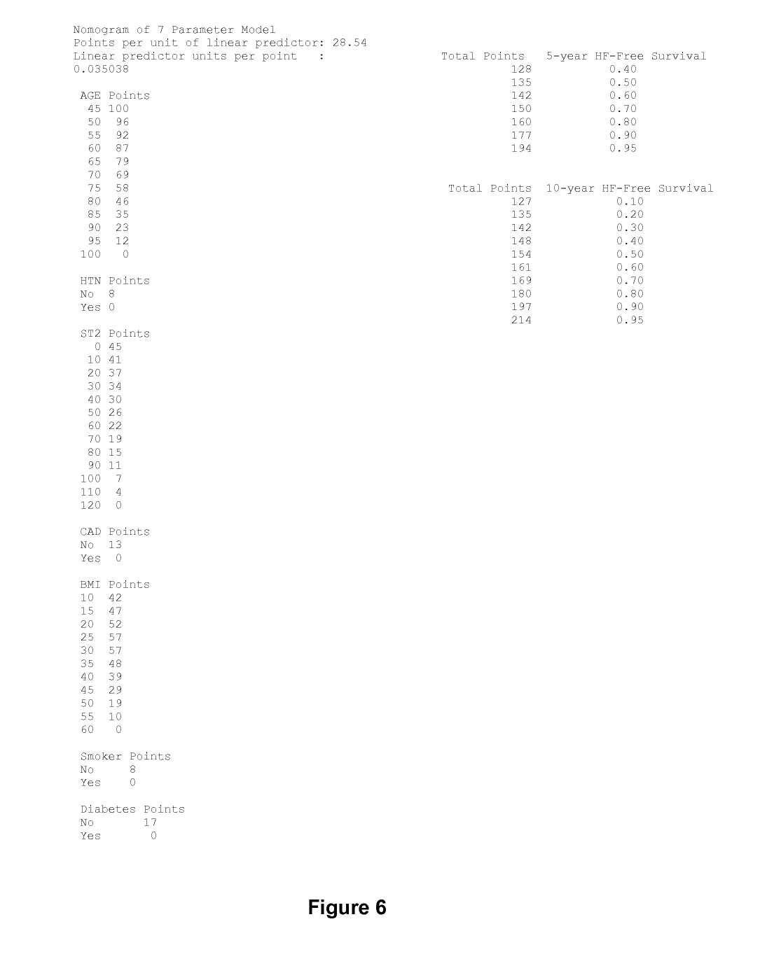

A summary of the analysis of Model 1 is shown in FIG. 1. The effect of each factor of soluble ST2, presence or absence of diabetes, presence or absence of hypertension, presence or absence of smoking, age, BMI, and presence or absence of coronary artery disease is shown in FIG. 2. A graph showing the partial χ² statistics of the association of soluble ST2, presence or absence of diabetes, presence or absence of hypertension, presence or absence of smoking, age, BMI, and presence or absence of coronary artery disease, with response is shown in FIG. 3, penalized for df. FIG. 4 is a bootstrap validation of the calibration curve. FIG. 5 is a nomogram for determining a subject's likelihood of heart failure-free survival within a period of 5 years or 10 years, based on the seven parameter model (Model 1). FIG. 6 is a summary of the nomogram based on the seven parameter model (Model 1).

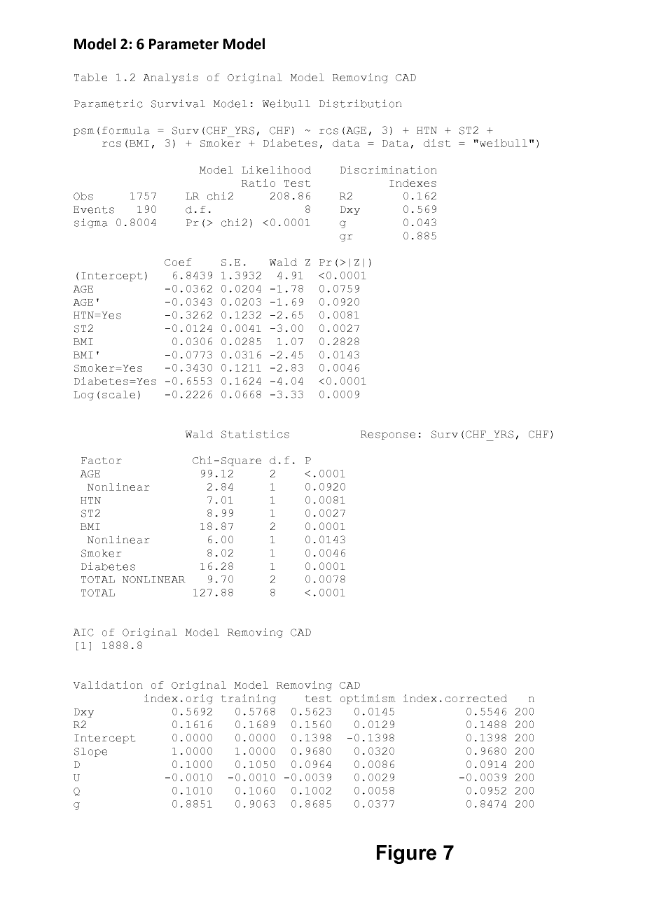

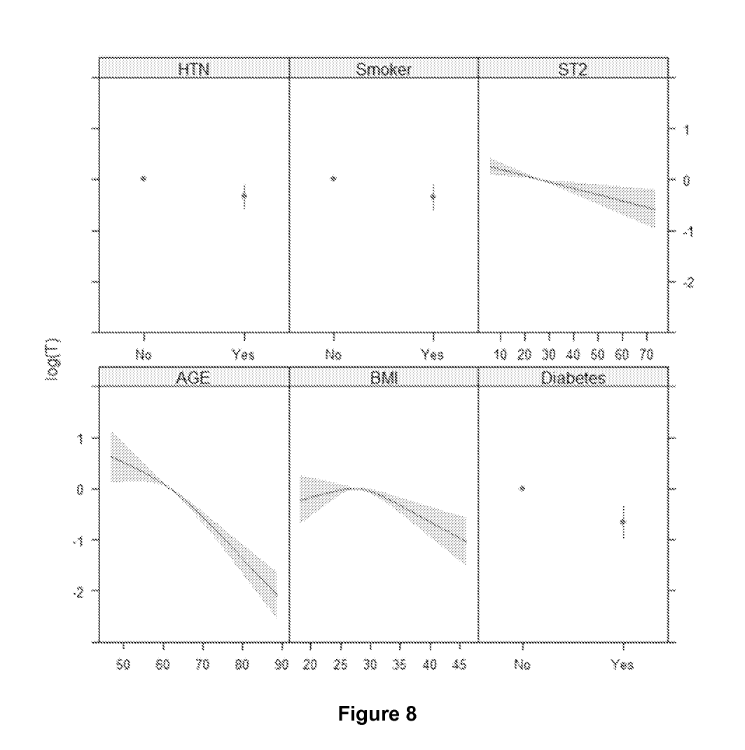

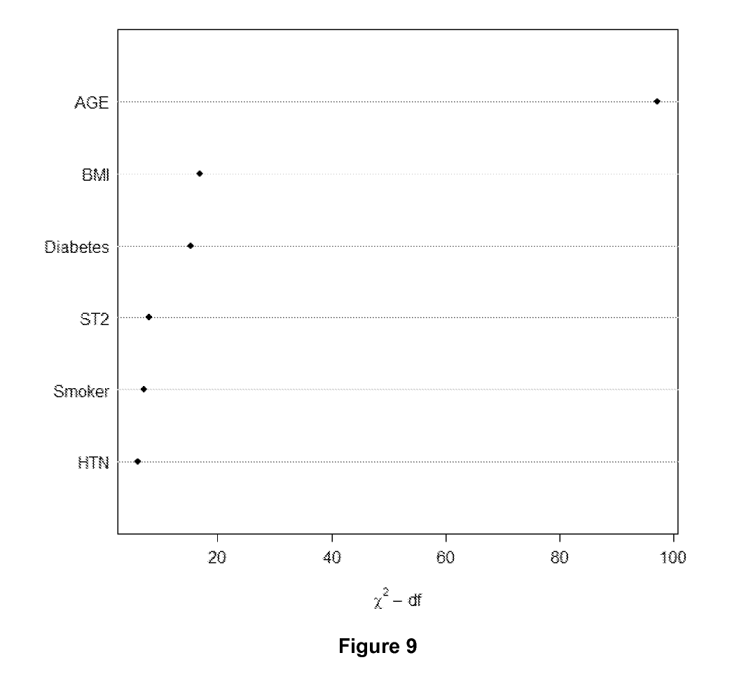

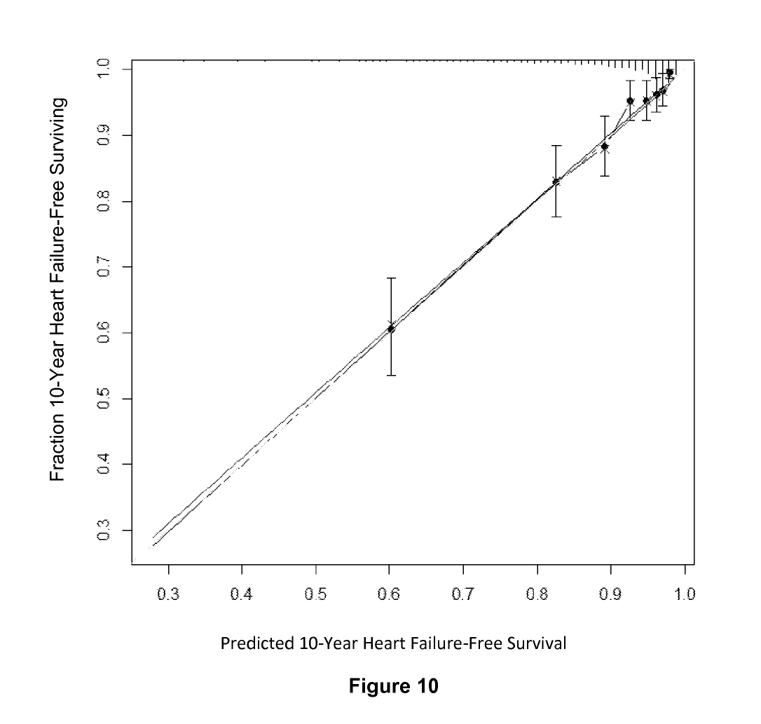

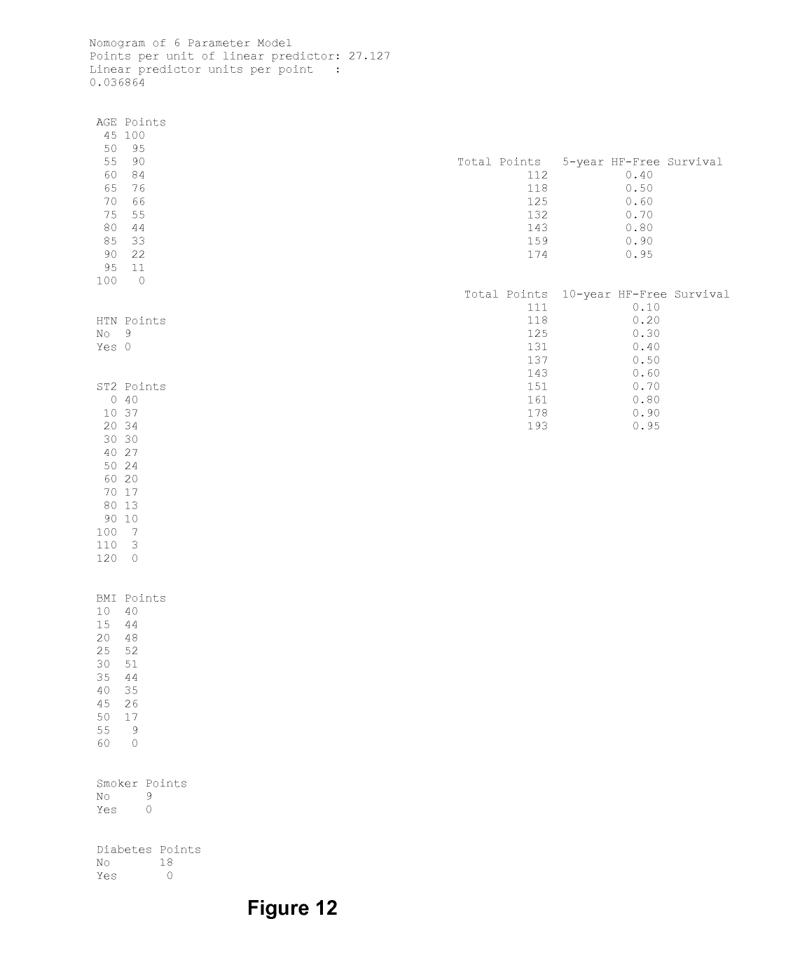

A summary of the analysis of Model 2 is shown in FIG. 7. The effect of each factor of presence or absence of hypertension, presence or absence of smoking behavior, serum soluble ST2 levels, age, body mass index, and presence or absence of diabetes is shown in FIG. 8. A graph showing the partial χ² statistics of the association of presence or absence of hypertension, presence or absence of smoking behavior, serum soluble ST2 levels, age, body mass index, and presence or absence of diabetes, with response is shown in FIG. 9, penalized for df. FIG. 10 is a bootstrap validation of the calibration curve. FIG. 11 is a nomogram for determining a subject's likelihood of heart failure-free survival within a time period of 5 years or 10 years, based on the seven parameter model (Model 2). FIG. 12 is a summary of the nomogram based on this six parameter model (Model 2).

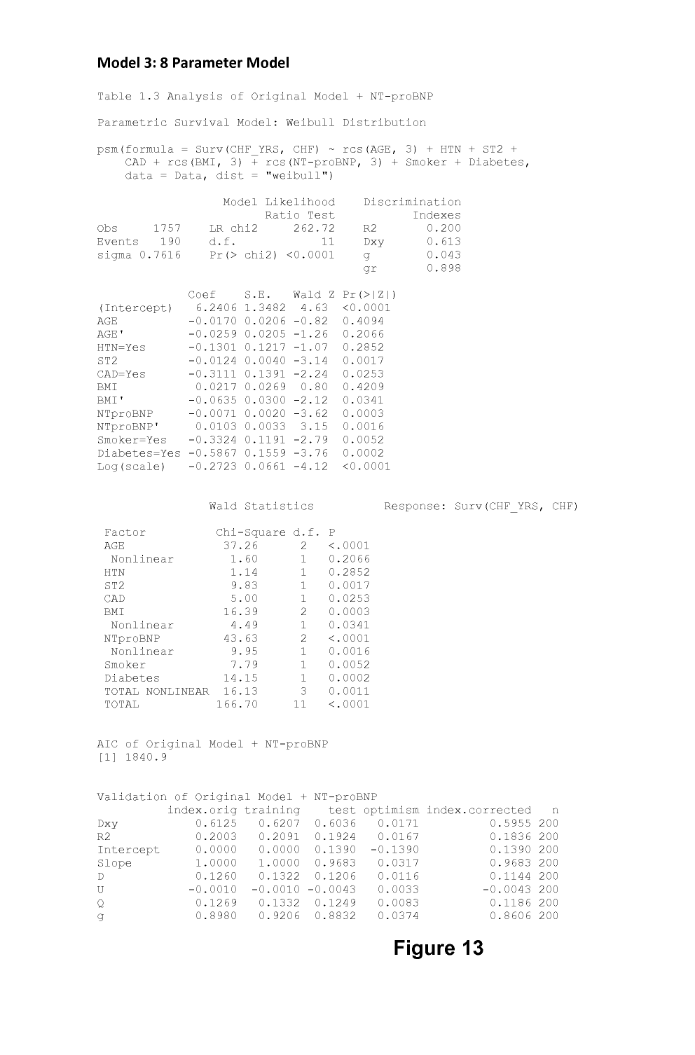

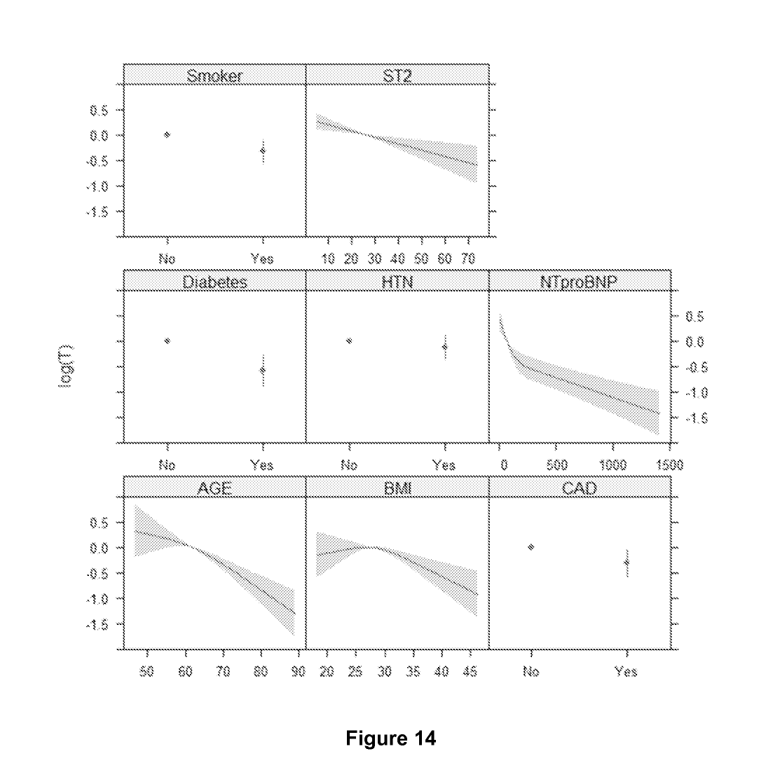

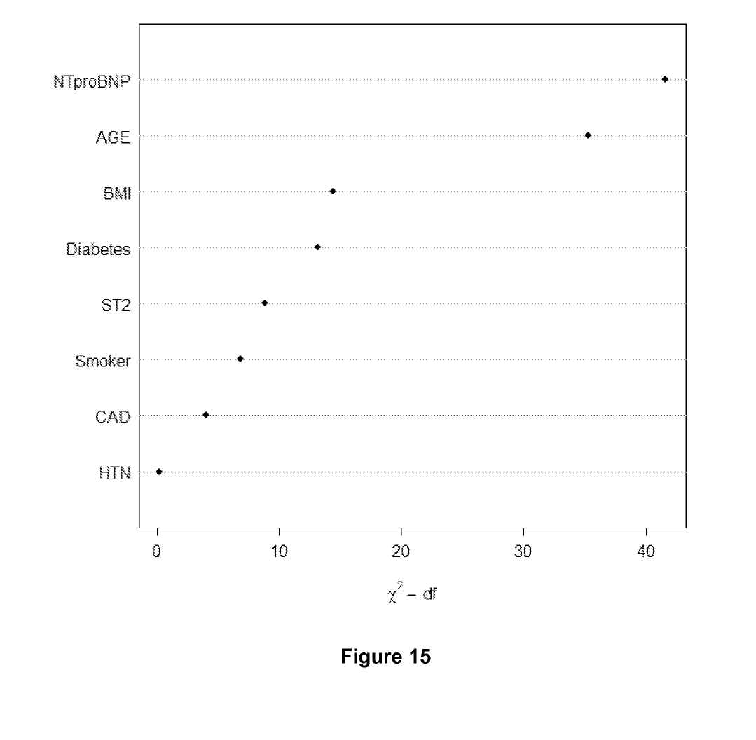

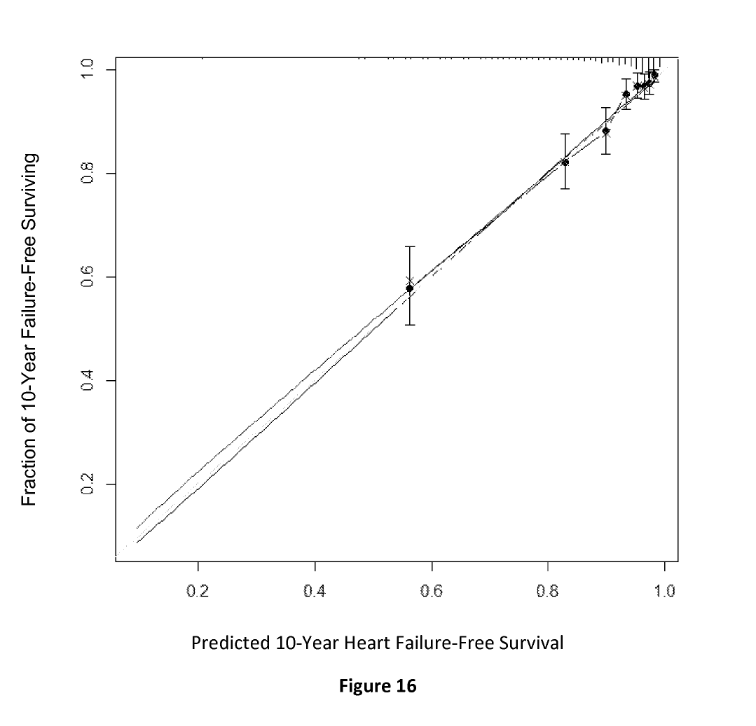

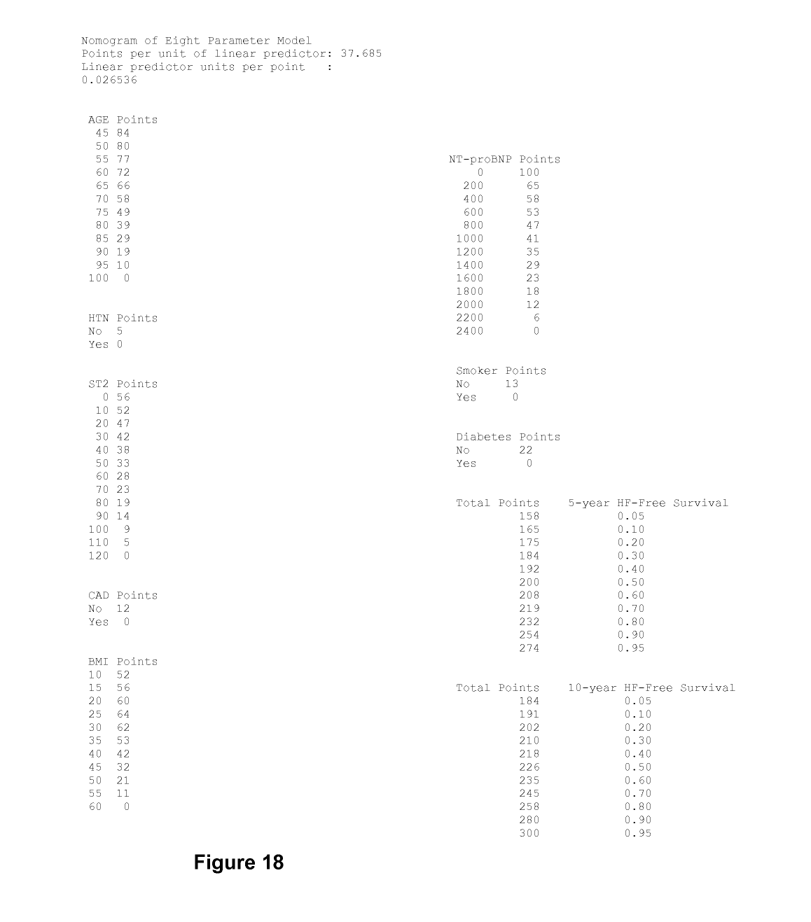

A summary of the analysis of Model 3 is shown in FIG. 13. The effect of each factor of presence or absence of smoking behavior, serum soluble ST2 levels, presence or absence of diabetes, presence or absence of hypertension, serum NT-proBNP levels, age, BMI, and presence or absence of coronary artery disease is shown in FIG. 14. A graph showing the partial χ² statistics of the association of presence or absence of smoking behavior, serum soluble ST2 levels, presence or absence of diabetes, presence or absence of hypertension, serum NT-proBNP levels, age, BMI, and presence or absence of coronary artery disease, with response is shown in FIG. 15, penalized for df. FIG. 16 is a bootstrap validation of the calibration curve. FIG. 17 is a nomogram for determining a subject's likelihood of heart failure-free survival within a period of 5 years or 10 years, based on the eight parameter model (Model 3). FIG. 18 is a summary of the nomogram based on this eight parameter model (Model 3).

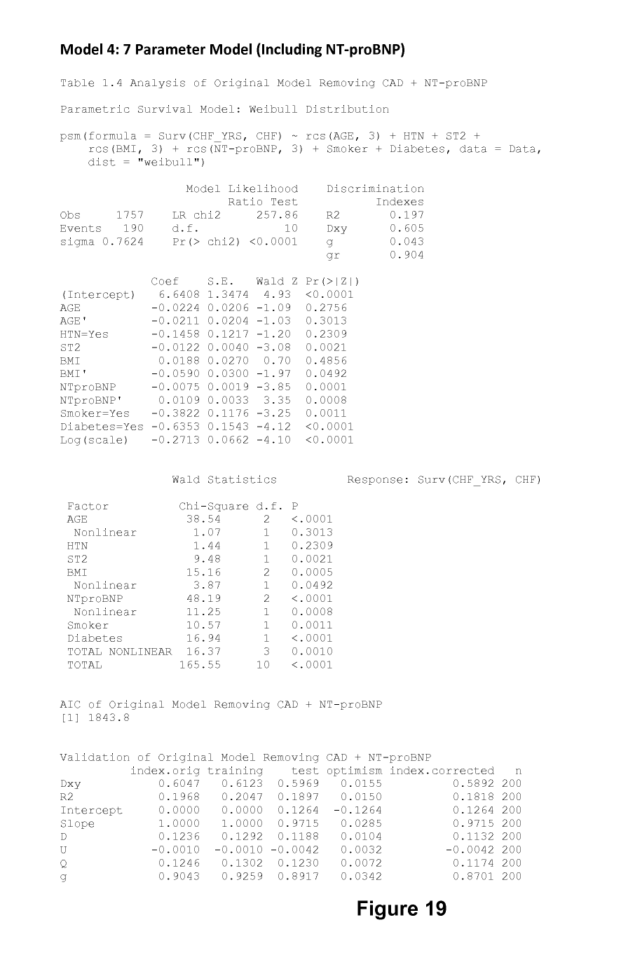

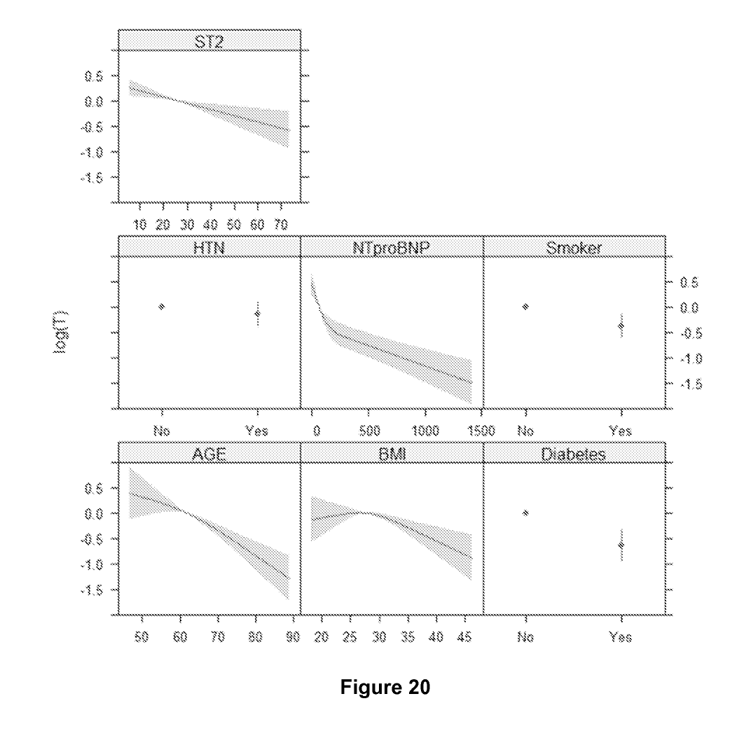

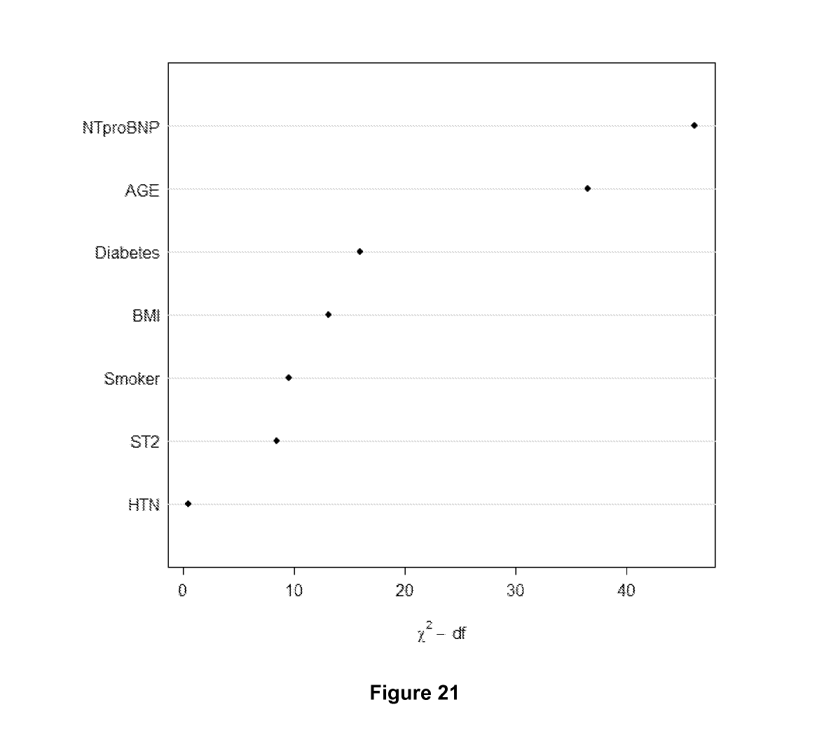

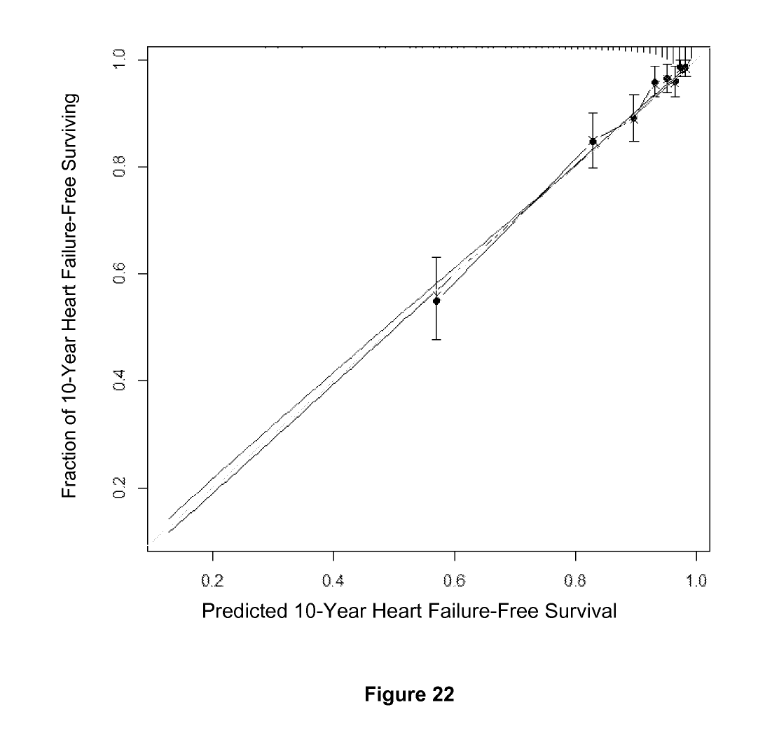

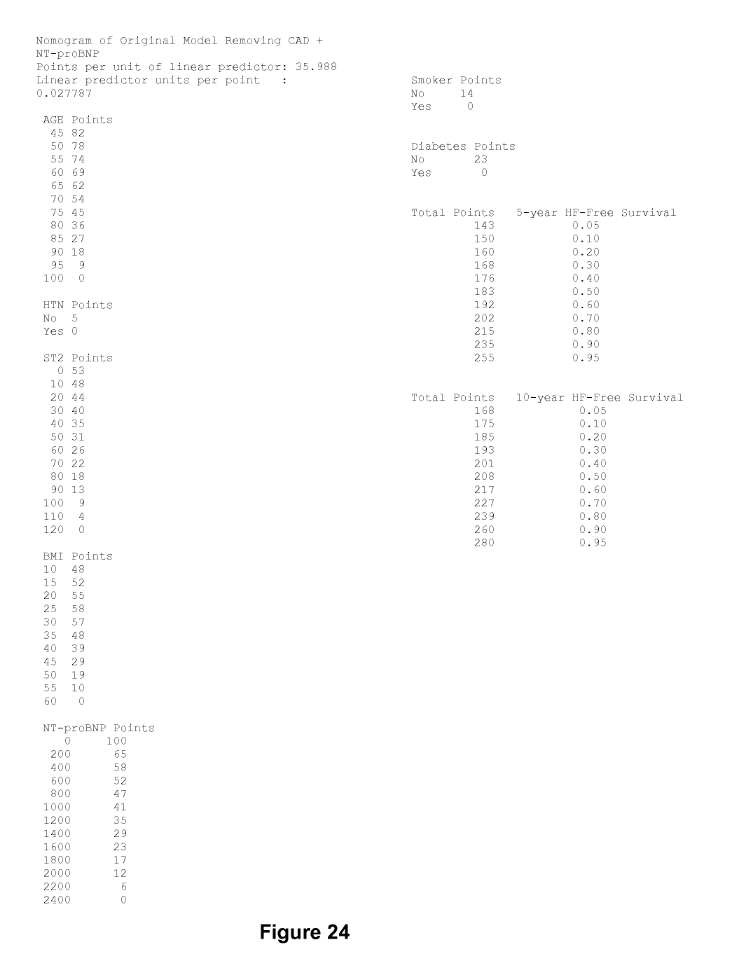

A summary of the analysis of Model 4 is shown in FIG. 19. The effect of each factor of presence or absence of serum soluble ST2 levels, presence or absence of hypertension, serum NT-proBNP levels, presence or absence of smoking behavior, age, BMI, and presence or absence of diabetes is shown in FIG. 20. A graph showing the partial χ² statistics of the association of presence or absence of serum soluble ST2 levels, presence or absence of hypertension, serum NT-proBNP levels, presence or absence of smoking behavior, age, BMI, and presence or absence of diabetes, with response is shown in FIG. 21, penalized for df. FIG. 22 is a bootstrap validation of the calibration curve. FIG. 23 is a nomogram for determining a subject's likelihood of heart failure-free survival within a time period of 5 years or 10 years, based on the eight parameter model (Model 4). FIG. 24 is a summary of the nomogram based on this seven parameter model (Model 4).

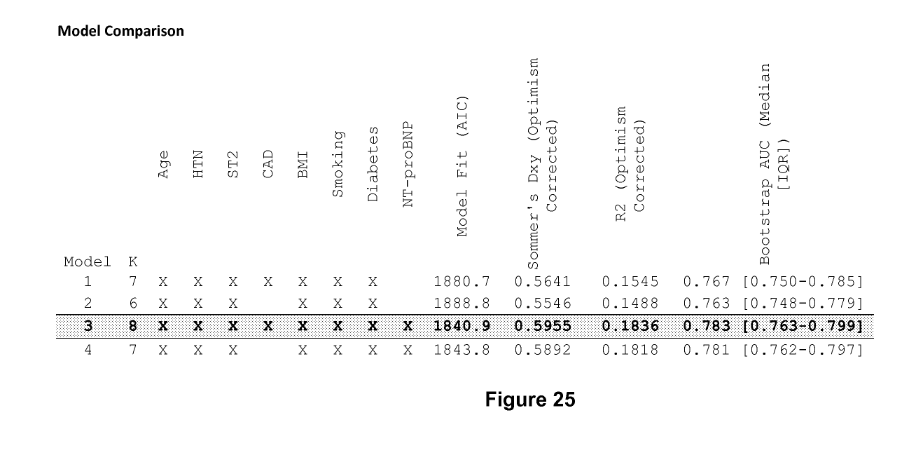

FIG. 25 is a chart providing a comparison of the accuracy of each of Models 1-4 (described in this example). The data show that Model 3 is the most accurate of the four models described herein.

An example of how to use the nomogram based on Model 2 is listed below.

### Model 1: 7 Parameter Model

1. Determine age and round to the nearest 5 years and estimate the number of points from the table below.

| AGE | Points |
|---|---|
| 45 | 100 |
| 50 | 96 |
| 55 | 92 |
| 60 | 87 |
| 65 | 79 |
| 70 | 69 |
| 75 | 58 |
| 80 | 46 |
| 85 | 35 |
| 90 | 23 |
| 95 | 12 |
| 100 | 0 |

2. Does the subject have Hypertension? If no, add 12 points.

3. Estimate subject's ST2 Concentration to the nearest 10 ng/mL and estimate the number of points from the table below.

| ST2 | Points |
|---|---|
| 0 | 45 |
| 10 | 41 |
| 20 | 37 |
| 30 | 34 |
| 40 | 30 |
| 50 | 26 |
| 60 | 22 |
| 70 | 19 |
| 80 | 15 |
| 90 | 11 |
| 100 | 7 |
| 110 | 4 |
| 120 | 0 |

4. Does the subject have cardiovascular disease? If no, add 13 points.

5. Determine BMI and round to the nearest 5 mg/kg² and estimate the number of points from the table below.

| BMI | Points |
|---|---|
| 10 | 42 |
| 15 | 47 |
| 20 | 52 |
| 25 | 57 |
| 30 | 57 |
| 35 | 48 |
| 40 | 39 |
| 45 | 29 |
| 50 | 19 |
| 55 | 10 |
| 60 | 0 |

6. Does the subject smoke? If no, add 8 points.

7. Does the subject have diabetes? If no, add 17 points

8. Add up the total number of points and 5-year heart failure-free survival can be determined from the following table.

| Total | 5-year HF-Free |
|---|---|
| Points | Survival |
| 128 | 0.40 |
| 135 | 0.50 |
| 142 | 0.60 |
| 150 | 0.70 |
| 160 | 0.80 |
| 177 | 0.90 |
| 194 | 0.95 |

9. 10-year heart failure-free survival can be determined from the following table.

| Total | 10-year HF-Free |
|---|---|
| Points | Survival |
| 127 | 0.10 |
| 135 | 0.20 |
| 142 | 0.30 |
| 148 | 0.40 |
| 154 | 0.50 |
| 161 | 0.60 |
| 169 | 0.70 |
| 180 | 0.80 |
| 197 | 0.90 |
| 214 | 0.95 |

### Example

A 54 year old smoker with hypertension but no evidence of cardiovascular disease comes in for an examination. The subject's BMI is determined to be 32 mg/kg² and ST2 concentration is measured as 42 ng/dL. Furthermore this subject has no evidence of diabetes. What is this subject's 5- and 10-year heart failure-free survival probability?

Answer:

1) Age Points=92

2) Smoking Points=0

3) Hypertension Points=0

4) Cardiovascular Disease Points=13

5) BMI Points=57

6) ST2 Points=30

7) Diabetes Points=17

Total Points=209

This subject's 5 year heart failure-free survival probability is >95% and the 10 year heart failure-free survival probability is between 90% and 95%.

An example of how to use the nomogram based on Model 2 is listed below.

### Model 2: 6 Parameter Model

1. Determine age and round to the nearest 5 years and estimate the number of points from the table below.

| AGE | Points |
|---|---|
| 45 | 100 |
| 50 | 95 |
| 55 | 90 |
| 60 | 84 |
| 65 | 76 |
| 70 | 66 |
| 75 | 55 |
| 80 | 44 |
| 85 | 33 |
| 90 | 22 |
| 95 | 11 |
| 100 | 0 |

2. Does the subject have hypertension? If no, add 9 points.

3. Estimate subjects ST2 concentration to the nearest 10 ng/mL and estimate the number of points from the table below.

| ST2 | Points |
|---|---|
| 0 | 40 |
| 10 | 37 |
| 20 | 34 |
| 30 | 30 |
| 40 | 27 |
| 50 | 24 |
| 60 | 20 |
| 70 | 17 |
| 80 | 13 |
| 90 | 10 |
| 100 | 7 |
| 110 | 3 |
| 120 | 0 |

4. Determine BMI and round to the nearest 5 mg/kg² and estimate the number of points from the table below.

| BMI | Points |
|---|---|
| 10 | 40 |
| 15 | 44 |
| 20 | 48 |
| 25 | 52 |
| 30 | 51 |
| 35 | 44 |
| 40 | 35 |
| 45 | 26 |
| 50 | 17 |
| 55 | 9 |
| 60 | 0 |

5. Does the subject smoke? If no, add 9 points.

6. Does the subject have diabetes? If no, add 18 points.

7. Add up the total number of points and 5-year heart failure-free survival can be determined from the following table.

| Total | 5-year HF-Free |
|---|---|
| Points | Survival |
| 112 | 0.40 |
| 118 | 0.50 |
| 125 | 0.60 |
| 132 | 0.70 |
| 143 | 0.80 |
| 159 | 0.90 |
| 174 | 0.95 |

8. 10-year heart failure-free survival can be determined from the following table.

| Total | 10-year HF-Free |
|---|---|
| Points | Survival |
| 111 | 0.10 |
| 118 | 0.20 |
| 125 | 0.30 |
| 131 | 0.40 |
| 137 | 0.50 |
| 143 | 0.60 |
| 151 | 0.70 |
| 161 | 0.80 |
| 178 | 0.90 |
| 193 | 0.95 |

### Example

A 54 year old smoker with hypertension but no evidence of cardiovascular disease comes in for an examination. The subject's BMI is determined to be 32 mg/kg² and ST2 concentration is measured as 42 ng/mL. Furthermore this subject has no evidence of diabetes. What is this subject's 5- and 10-year heart failure-free survival probability?

Answer:

1) Age Points=95

2) Smoking Points=0

3) Hypertension Points=0

4) BMI Points=51

5) ST2 Points=27

6) Diabetes Points=18

Total Points=191

This subject's 5-year heart failure-free survival probability is >95% and the 10-year heart failure-free survival probability is between 90% and 95%.

### Example

A 65 year old diabetic non-smoker with hypertension comes in for an examination. The subject's BMI is determined to be 36 mg/kg² and ST2 concentration is measured as 56 ng/mL. What is this subject's 5 and 10 year heart failure-free survival probability?

Answer:

1) Age Points=76

2) Diabetes Points=0

3) Smoking Points=9

4) Hypertension Points=0

5) BMI Points=44

6) ST2 Points=20

Total Points=149

This subject's 5-year heart failure-free survival probability is between 80% and 90% and the 10-year heart failure-free survival probability is between 60% and 70%.

An example of how to use the nomogram based on Model 3 is listed below.

### Model 3: 8 Parameter Model

1. Determine age and round to the nearest 5 years and estimate the number of points from the table below.

| AGE | Points |
|---|---|
| 45 | 84 |
| 50 | 80 |
| 55 | 77 |
| 60 | 72 |
| 65 | 66 |
| 70 | 58 |
| 75 | 49 |
| 80 | 39 |
| 85 | 29 |
| 90 | 19 |
| 95 | 10 |
| 100 | 0 |

2. Does the subject have hypertension? If no, add 5 points.

3. Estimate subject's ST2 Concentration to the nearest 10 ng/mL and estimate the number of points from the table below.

| ST2 | Points |
|---|---|
| 0 | 56 |
| 10 | 52 |
| 20 | 47 |
| 30 | 42 |
| 40 | 38 |
| 50 | 33 |
| 60 | 28 |
| 70 | 23 |
| 80 | 19 |
| 90 | 14 |
| 100 | 9 |
| 110 | 5 |
| 120 | 0 |

4. Does the subject have cardiovascular disease? If no, add 12 points.

5. Determine BMI and round to the nearest 5 mg/kg² and estimate the number of points from the table below.

| BMI | Points |
|---|---|
| 10 | 52 |
| 15 | 56 |
| 20 | 60 |
| 25 | 64 |
| 30 | 62 |
| 35 | 53 |
| 40 | 42 |
| 45 | 32 |
| 50 | 21 |
| 55 | 11 |
| 60 | 0 |

6. Determine NT-proBNP to the nearest 200 pg/mL and estimate the number of points from the table below.

| NT-proBNP | Points |
|---|---|
| 0 | 100 |
| 200 | 65 |
| 400 | 58 |
| 600 | 53 |
| 800 | 47 |
| 1000 | 41 |
| 1200 | 35 |
| 1400 | 29 |
| 1600 | 23 |
| 1800 | 18 |
| 2000 | 12 |
| 2200 | 6 |
| 2400 | 0 |

7. Does the subject smoke? If no, add 13 points.

8. Does the subject have diabetes? If no, add 22 points.

9. Add up the total number of points and 5-year heart failure-free survival can be determined from the following table.

| Total | 5 year HF-Free |
|---|---|
| Points | Survival |
| 158 | 0.05 |
| 165 | 0.10 |
| 175 | 0.20 |
| 184 | 0.30 |
| 192 | 0.40 |
| 200 | 0.50 |
| 208 | 0.60 |
| 219 | 0.70 |
| 232 | 0.80 |
| 254 | 0.90 |
| 274 | 0.95 |
| 275 | |

10. 10-year heart failure-free survival can be determined from the following table.

| Total | 10 Year HF-Free |
|---|---|
| Points | Survival |
| 184 | 0.05 |
| 191 | 0.10 |
| 202 | 0.20 |
| 210 | 0.30 |
| 218 | 0.40 |
| 226 | 0.50 |
| 235 | 0.60 |
| 245 | 0.70 |
| 258 | 0.80 |
| 280 | 0.90 |
| 300 | 0.95 |

### Example

A 54 year old smoker with hypertension but no evidence of cardiovascular disease comes in for an examination. The subject's BMI is determined to be 32 mg/kg² and ST2 concentration is measured as 42 ng/mL and NT-proBNP is measured at 1600 pg/mL. Furthermore this subject has no evidence of diabetes. What is this subject's 5- and 10-year heart failure-free survival probability?

Answer:

1) Age Points=77

2) Smoking Points=0

3) Hypertension Points=0

4) BMI Points=62

5) ST2 Points=38

6) NT-proBNP Points=23

7) Diabetes Points=22

Total Points=222

This subject's 5-year heart failure-free survival probability is between 70% and 80% and the 10-year heart failure-free survival probability is between 40% and 50%.

An example of how to use the nomogram based on Model 4 is listed below.

### Model 4: 7 Parameter Model (Including NT-proBNP)

1. Determine age and round to the nearest 5 years and estimate the number of points from the table below.

| AGE | Points |
|---|---|
| 45 | 82 |
| 50 | 78 |
| 55 | 74 |
| 60 | 69 |
| 65 | 62 |
| 70 | 54 |
| 75 | 45 |
| 80 | 36 |
| 85 | 27 |
| 90 | 18 |
| 95 | 9 |
| 100 | 0 |

2. Does the subject have hypertension? If no, add 5 points.

3. Estimate subject's ST2 concentration to the nearest 10 ng/mL and estimate the number of points from the table below.

| ST2 | Points |
|---|---|
| 0 | 53 |
| 10 | 48 |
| 20 | 44 |
| 30 | 40 |
| 40 | 35 |
| 50 | 31 |
| 60 | 26 |
| 70 | 22 |
| 80 | 18 |
| 90 | 13 |
| 100 | 9 |
| 110 | 4 |
| 120 | 0 |

4. Determine BMI and round to the nearest 5 mg/kg² and estimate the number of points from the table below.

| BMI | Points |
|---|---|
| 10 | 48 |
| 15 | 52 |
| 20 | 55 |
| 25 | 58 |
| 30 | 57 |
| 35 | 48 |
| 40 | 39 |
| 45 | 29 |
| 50 | 19 |
| 55 | 10 |
| 60 | 0 |

5. Determine NT-proBNP to the nearest 200 pg/mL and estimate the number of points from the table below.

| NT-proBNP | Points |
|---|---|
| 0 | 100 |
| 200 | 65 |
| 400 | 58 |
| 600 | 52 |
| 800 | 47 |
| 1000 | 41 |
| 1200 | 35 |
| 1400 | 29 |
| 1600 | 23 |
| 1800 | 17 |
| 2000 | 12 |
| 2200 | 6 |
| 2400 | 0 |

6. Does the subject smoke? If no, add 14 points.

7. Does the subject have diabetes? If no, add 23 points.

8. Add up the total number of points and 5-year heart failure-free survival can be determined from the following table.

| Total | 5 year HF-Free |
|---|---|
| Points | Survival |
| 143 | 0.05 |
| 150 | 0.10 |
| 160 | 0.20 |
| 168 | 0.30 |
| 176 | 0.40 |
| 183 | 0.50 |
| 192 | 0.60 |
| 202 | 0.70 |
| 215 | 0.80 |
| 235 | 0.90 |
| 255 | 0.95 |

9. 10-year heart failure-free survival can be determined from the following table.

| Total | 10 year HF-Free |
|---|---|
| Points | Survival |
| 168 | 0.05 |
| 175 | 0.10 |
| 185 | 0.20 |
| 193 | 0.30 |
| 201 | 0.40 |
| 208 | 0.50 |
| 217 | 0.60 |
| 227 | 0.70 |
| 239 | 0.80 |
| 260 | 0.90 |
| 280 | 0.95 |

### Example

A 54 year old smoker with hypertension but no evidence of cardiovascular disease comes in for an examination. The subject's BMI is determined to be 32 mg/kg² and ST2 concentration is measured as 42 ng/mL and NT-proBNP is measured at 1600 pg/mL. Furthermore this subject has no evidence of diabetes. What is this subject's 5- and 10-year heart failure-free survival probability?

Answer:

1) Age Points=74

2) Smoking Points=0

3) Hypertension Points=0

4) BMI Points=57

5) ST2 Points=35

6) NT-proBNP Points=23

7) Diabetes Points=23

Total Points=212

This subject's 5-year heart failure-free survival probability is between 70% and 80% and the 10-year heart failure-free survival probability is between 50% and 60%.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining the risk of developing heart failure within a specific time period in a subject not diagnosed or presenting with heart failure, the method comprising:
(a) providing a set of factors relating to the subject's health comprising: presence or absence of hypertension in the subject, smoking or non-smoking behavior of the subject, serum level of soluble ST2 in the subject, age of the subject, body mass index of the subject, and presence or absence of diabetes in the subject;
(b) determining a separate point value for each of the provided factors in (a);
(c) adding the separate point values for each of the provided factors in (b) together to yield a total points value; and
(d) determining the subject's risk of developing heart failure within a specific time period by correlating the total points value in (c) with a value on a predictor scale of risk of developing heart failure within the specific time period based on the set of factors obtained from a population of subjects not diagnosed or presenting with heart failure.

2.-4. (canceled)

5. The method of claim 1, wherein the providing in (a) comprises obtaining the set of factors from the subject's recorded clinical information through a computer software program.

6. (canceled)

7. The method of claim 1, wherein the providing step in (a) comprises the manual entry of the set of factors into a website interface or a software program.

8. The method of claim 1, further comprising determining one or more of the set of factors in (a) in a subject.

9. (canceled)

10. The method of claim 1, wherein one or more of the determining in (b), the adding in (c), and the determining in (d) is performed using a software program.

11. The method of claim 1, further comprising:
(e) comparing the determined risk of developing heart failure within the specific time period to a predetermined risk value;
(f) identifying a subject whose determined risk of developing heart failure within the specific time period is elevated as compared to the predetermined risk value; and
(g) administering a treatment for reducing the risk of developing heart failure to the identified subject.

12. The method of claim 11, wherein one or both of the comparing in (e) and the identifying in (f) are performed using a software program.

13. A method for determining the efficacy of a treatment for reducing the risk of developing heart failure in a subject, the method comprising:
(a) providing a set of factors relating to the subject's health at a first time point comprising: presence or absence of hypertension in the subject, smoking or non-smoking behavior of the subject, serum level of soluble ST2 in the subject, age of the subject, body mass index of the subject, and presence or absence of diabetes in the subject;
(b) determining a separate point value for each of the provided factors in (e);
(c) adding the separate point values for each of the provided factors in (f) together to yield a total points value;
(d) determining the subject's risk of developing heart failure within the specific time period at the first time point by correlating the total points value of (c) with a value on a predictor scale of risk of developing heart failure within the specific time period based on the set of factors obtained from a population of subjects not diagnosed or presenting with heart failure;
(e) providing a set of factors relating to the subject's health at a second time point comprising: presence or absence of hypertension in the subject, smoking or non-smoking behavior of the subject, serum level of soluble ST2 in the subject, age of the subject, body mass index of the subject, and presence or absence of diabetes in the subject;
(f) determining a separate point value for each of the provided factors in (e);
(g) adding the separate point values for each of the provided factors in (f) to yield a total points value;
(h) determining the subject's risk of developing heart failure within the specific time period at the second time point by correlating the total points value of (g) with a value on a predictor scale of risk of developing heart failure within the specific time period based on the set of factors obtained from a population of subjects not diagnosed or presenting with heart failure, wherein the second time point is after the first time point, and the subject has received at least two doses of a treatment after the first time point and before the second time point;
(i) comparing the subject's risk of developing heart failure within the specific time period determined at the second time point to the subject's risk of developing heart failure within the specific time period determined at the first time point; and
(j) identifying the treatment administered to a subject having a decreased risk of developing heart failure within the specific time period determined at the second time point as compared the subject's risk of developing heart failure within the specific time period determined at the first time point as being effective for reducing the risk of developing heart failure, or
identifying the treatment administered to a subject having an elevated or about the same risk of developing heart failure within the specific time period determined at the second time point as compared to the subject's risk of developing heart failure within the specific time period determined at the first time point as not being effective for reducing the risk of developing heart failure.

14. (canceled)

15. The method of claim 13, further comprising administering a treatment for reducing the risk of developing heart failure to the identified subject after the first time point and before the second time point.

16. A method for selecting a treatment for a subject not diagnosed or presenting with heart failure, the method comprising:
(a) providing a set of factors relating to the subject's health at a first time point comprising: presence or absence of hypertension in the subject, smoking or non-smoking behavior of the subject, serum level of soluble ST2 in the subject, age of the subject, body mass index of the subject, and presence or absence of diabetes in the subject;
(b) determining a separate point value for each of the provided factors in (a);
(c) adding the separate point values for each of the provided factors in (b) together to yield a total points value;
(d) determining the subject's risk of developing heart failure within a specific time period at the first time point by correlating the total points value of (c) with a value on a predictor scale of risk of developing heart failure within the specific time period based on the set of factors obtained from a population of subjects not diagnosed or presenting with heart failure;
(e) providing a set of factors relating to the subject's health at a second time point comprising: presence or absence of hypertension in the subject, smoking or non-smoking behavior of the subject, serum level of soluble ST2 in the subject, age of the subject, body mass index of the subject, and presence or absence of diabetes in the subject;
(f) determining a separate point value for each of the provided factors in (e);
(g) adding the separate point values for each of the provided factors in (f) together to yield a total points value;
(h) determining the subject's risk of developing heart failure within the specific time period at the second time point by correlating the total points value of (g) with a value on a predictor scale of risk of developing heart failure within the specific time period based on the set of factors obtained from a population of subjects not diagnosed or presenting with heart failure, wherein the second time point is after the first time point, and the subject has received a treatment after the first time point and before the second time point;
(i) comparing the subject's risk of developing heart failure within the specific time period determined at the second time point to the subject's risk of developing heart failure within the specific time period determined at the first time point; and
(j) identifying a subject having an elevated or about the same risk of developing heart failure within the specific time period determined at the second time point as compared to the subject's risk of developing heart failure within the specific time period determined at the first time point, and selecting an alternate treatment for the subject, or
identifying a subject having a reduced risk of developing heart failure within the specific time period determined at the second time point as compared to the subject's risk of developing heart failure within the specific time period determined at the first time point, and selecting the same treatment for the subject.

17.-19. (canceled)

20. The method of claim 16, further comprising administering the selected treatment to the identified subject after the second time point.

21. A nomogram for the graphic representation of a quantitative probability that a subject not diagnosed or presenting with heart failure will develop heart failure within a specific time period comprising the following elements (a), (b), and (c) depicted on a two-dimensional support:
(a) a plurality of scales comprising a presence of hypertension scale, a smoking behavior scale, a serum level of soluble ST2 scale, an age of the subject scale, a body mass index scale, and a presence of diabetes scale;
(b) a point scale; and
(c) a predictor scale,
wherein each of the plurality of scales of (a) has values, the plurality of scales of (a) is depicted on the two-dimensional support with respect to the point scale in (b), such that the values on each of the plurality of scales can be correlated with values on the point scale, and the predictor scale contains information correlating a sum of each of correlated values on the point scale to the quantitative probability that a subject not diagnosed or presenting with heart failure will develop heart failure within a specific time period.

22.-24. (canceled)

25. A method of determining the quantitative probability that a subject not diagnosed or presenting with heart failure will develop heart failure within a specific time period comprising the use of the nomogram of claim 21.

26. A computer-implemented method comprising:
accessing a set of factors relating to a subject's health, the set of factors representing one or more of: presence or absence of hypertension in the subject, smoking or non-smoking behavior of the subject, presence or absence of coronary artery disease in the subject, serum level of soluble ST2 in the subject, serum level of N-terminal pro-brain natriuretic peptide (NT-proBNP) in the subject, age of the subject, body mass index of the subject, and presence or absence of diabetes in the subject;
determining, using a processor, a separate point value for each factor in the set of factors;
determining a total points value as a function of the separate point values; and
determining the subject's risk of the subject developing heart failure within a specific time period by correlating the total points value with a value on a predictor scale of risk of developing heart failure within the specific time period, respectively, wherein the predictor scale is based on a set of factors obtained from a population of subjects not diagnosed or presenting with heart failure.

27.-30. (canceled)

31. The method of claim 1, wherein the provided factors in (a) further comprises presence or absence of coronary artery disease in the subject.

32. The method of claim 1, wherein the provided in factors in (a) further comprises presence or absence of coronary artery disease in the subject and serum level of N-terminal pro-brain natriuretic peptide (NT-proBNP) in the subject.

33. The method of claim 1, wherein the provided factors in (a) further comprises serum level of N-terminal pro-brain natriuretic peptide (NT-proBNP) in the subject.

34. The nomogram of claim 21, wherein the plurality of scales of (a) further comprises a presence of coronary artery disease scale.

35. The nomogram of claim 21, wherein the plurality of scales of (a) further comprises a presence of coronary artery disease scale and a serum level of N-terminal pro-brain natriuretic peptide (NT-proBNP) scale.

36. The nomogram of claim 21, wherein the plurality of scales of (a) further comprises a serum level of N-terminal pro-brain natriuretic peptide (NT-proBNP) scale.
